SEQUENCE LISTING (57 sequences)

SEQ ID NO: 1 - DNA, 906 bp, Umbellularia californica
  misc_feature (1)..(906): synUcTE (an acyl-CoA thioesterase) gene (codon- optimized)
atgactctagagtggaaaccgaaaccaaaactgcctcaactgctggatgatcacttcggtctgcacggtctggtgtttcgtcgtactttcgcaattcgttcttatgaagtgggtccagatcgttctacctccatcctggccgtcatgaaccacatgcaggaagccaccctgaatcacgcgaaatctgttggtatcctgggtgatggtttcggcactactctggaaatgtctaaacgtgacctgatgtgggtagtgcgtcgcacccacgtagcagtagagcgctaccctacttggggtgacactgtggaagtcgagtgttggattggcgcgtccggtaacaatggtatgcgtcgcgattttctggtccgtgactgtaaaacgggcgaaatcctgacgcgttgcacctccctgagcgttctgatgaacacccgcactcgtcgcctgtctaccatcccggacgaagtgcgcggtgagatcggtcctgctttcatcgataacgtggcagttaaagacgacgaaatcaagaaactgcaaaaactgaacgactccaccgcggactacatccagggcggtctgactccgcgctggaacgacctggatgttaatcagcatgtgaacaacctgaaatacgttgcttgggtcttcgagactgtgccggacagcattttcgaaagccatcacatttcctcttttactctggagtaccgtcgcgaatgtactcgcgactccgttctgcgcagcctgaccaccgtaagcggcggttctagcgaggcaggtctggtctgcgaccatctgctgcaactggaaggcggctccgaagtcctgcgtgcgcgtacggagtggcgtccaaagctgacggattctttccgcggcatctccgtaattccggcggaacctcgtgtttaa

SEQ ID NO: 2 - DNA, 159 bp, Artificial Sequence
  tac promotor for synUcTE expression
ggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggagg

SEQ ID NO: 3 - DNA, 4977 bp, Artificial Sequence
  Vector pJ294[Ptac-synUcTE]
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtgggggcggccgcaaattcagtaagcagaaagtcaaaagcctccgaccggaggcttttgactaaagcttaaacacgaggttccgccggaattacggagatgccgcggaaagaatccgtcagctttggacgccactccgtacgcgcacgcaggacttcggagccgccttccagttgcagcagatggtcgcagaccagacctgcctcgctagaaccgccgcttacggtggtcaggctgcgcagaacggagtcgcgagtacattcgcgacggtactccagagtaaaagaggaaatgtgatggctttcgaaaatgctgtccggcacagtctcgaagacccaagcaacgtatttcaggttgttcacatgctgattaacatccaggtcgttccagcgcggagtcagaccgccctggatgtagtccgcggtggagtcgttcagtttttgcagtttcttgatttcgtcgtctttaactgccacgttatcgatgaaagcaggaccgatctcaccgcgcacttcgtccgggatggtagacaggcgacgagtgcgggtgttcatcagaacgctcagggaggtgcaacgcgtcaggatttcgcccgttttacagtcacggaccagaaaatcgcgacgcataccattgttaccggacgcgccaatccaacactcgacttccacagtgtcaccccaagtagggtagcgctctactgctacgtgggtgcgacgcactacccacatcaggtcacgtttagacatttccagagtagtgccgaaaccatcacccaggataccaacagatttcgcgtgattcagggtggcttcctgcatgtggttcatgacggccaggatggaggtagaacgatctggacccacttcataagaacgaattgcgaaagtacgacgaaacaccagaccgtgcagaccgaagtgatcatccagcagttgaggcagttttggtttcggtttccactctagagtcattttataatcctccttgtaaacaaaattatttctagagggaaaccgttgtggaattgtgagcgctcacaattccacatccacacattatacgagccgatgattaattgtcaacagcgtcgatcactgtgcatgaagctcgtaattgttatccgctcacaattggatccaaaatgaagggaagttcctatactttctagagaataggaacttctatagggagtcgaataagggcgacacaaaaggtattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccaggatgcctggcagttccctactctcgccgctgcgctcggtcgttcggctgcgggacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgctttt

SEQ ID NO: 4 - DNA, 882 bp, Homo sapiens
  misc_feature (1)..(882): hGLYAT2 (an amino acid N-acyl transferase) codon optimised for E.coli
atgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgt

SEQ ID NO: 5 - DNA, 864 bp, Homo sapiens
  misc_feature (1)..(864): hGLYAT3 (another amino acid N-acyl transferase) (codon optimised for E.coli)
atgctggttctgaattgctctactaaactgctgatcctggagaaaatgctgaaatcctgtttcccggaatctctgaaagtttatggtgcggtaatgaatatcaaccgtggtaatccgtttcaaaaagaagttgttctggactcttggccagatttcaaggcggttatcacccgtcgtcagcgtgaagcggaaaccgacaacctggaccactataccaacgcgtatgcggttttctataaagatgttcgcgcgtaccgtcagctgctggaagaatgcgatgttttcaactgggaccaggtttttcagatccaaggtctgcaatccgagctgtacgacgtttctaaggccgtggcgaactctaaacaactgaacatcaagctgacctctttcaaagcggttcatttctctccggtatccagcctgccggacacctccttcctgaaaggtccgtctccgcgtctgacctacctgtctgttgcgaatgcggatctgctgaaccgtacttggtcccgtggtggcaacgaacagtgcctgcgttacattgctaacctgatctcttgcttcccgagcgtctgcgttcgtgatgaaaaaggtaacccagtatcttggtctatcaccgaccagttcgcgaccatgtgtcatggctacaccctgcctgaacaccgtcgtaagggttactctcgtctggttgcgctgaccctggcgcgtaagctgcagtctcgtggttttccgtcccagggtaacgtcctggacgacaacactgcgtccatctccctgctgaagtccctgcatgccgaattcctgccgtgccgtttccaccgtctgattctgaccccagcgaccttctctggtctgccgcacctg

SEQ ID NO: 6 - DNA, 1686 bp, Escherichia coli
  misc_feature (1)..(1686): fadD (an acyl-CoA synthetase)
ttgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctga

SEQ ID NO: 7 - DNA, 7138 bp, artificial sequence
  Vector pCDF[atfA1_Ab(co_Ec)-fadD_Ec]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgaaagcgctgtccccggtggatcagctgttcctgtggctggagaaacgtcagcaaccgatgcatgttggtggtctgcagctgtttagcttcccagagggtgcgggtcctaagtacgttagcgagttggctcagcaaatgcgtgactactgtcacccggtggccccgttcaatcagcgcctgacccgtcgcctgggtcaatactattggacgcgcgacaaacaatttgacattgaccaccattttcgccatgaagcgctgccgaagccgggtcgcattcgcgaactgctgtccttggttagcgcggagcacagcaatctgttggaccgcgagcgccctatgtgggaagcgcacctgattgagggcatccgtggccgccaattcgctttgtactataagatccaccatagcgtgatggatggcatcagcgctatgcgcattgcgtctaaaacgctgagcaccgacccgtctgagcgcgaaatggcaccagcatgggccttcaataccaagaaacgtagccgtagcctgccttccaatccagttgatatggcgagcagcatggcccgtctgacggcttctatcagcaaacaggcggcgaccgttccgggtctggctcgtgaagtgtacaaggtcacccagaaagctaagaaggatgagaactacgtgagcatttttcaagcgccggataccatcttgaacaacaccattaccggttcccgtcgttttgcagcacagagctttccgctgccgcgtctgaaagttatcgcgaaggcgtataactgcaccatcaacaccgtcgtcctgtctatgtgtggccacgcgctgcgtgaatatctgattagccaacacgcactgccggatgagccgctgatcgcgatggtgccgatgagcctgcgtcaggacgacagcacgggtggtaatcagattggcatgatcctggccaacctgggcacccatatctgcgatccggcgaatcgtttgcgtgttattcacgactccgtcgaggaagcgaaatcgcgtttcagccagatgagcccggaggagatcctgaactttaccgcactgactatggccccgacgggcttgaatctgctgaccggcctggcaccgaaatggcgtgcgttcaacgtcgtcattagcaacattccgggtccgaaggaaccgctgtattggaatggcgcccagctgcaaggtgtgtacccggtaagcattgcgttggaccgtattgcactgaacatcacgctgactagctacgtggatcagatggagttcggtctgatcgcatgccgtcgcacgctgccgagcatgcaacgtctgctggattatctggaacaaagcattcgtgagttggaaatcggtgccggcatcaagtaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 8 - DNA, 6649 bp, artificial sequence
  Vector pCDF{Ptac}[hGLYAT2(co_Ec)-fadD_Ec]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgttaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 9 - DNA, 6631 bp, Artificial Sequence
  Vector pCDF{Ptac}[hGLYAT3(co_Ec)-fadD_Ec]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgaattgctctactaaactgctgatcctggagaaaatgctgaaatcctgtttcccggaatctctgaaagtttatggtgcggtaatgaatatcaaccgtggtaatccgtttcaaaaagaagttgttctggactcttggccagatttcaaggcggttatcacccgtcgtcagcgtgaagcggaaaccgacaacctggaccactataccaacgcgtatgcggttttctataaagatgttcgcgcgtaccgtcagctgctggaagaatgcgatgttttcaactgggaccaggtttttcagatccaaggtctgcaatccgagctgtacgacgtttctaaggccgtggcgaactctaaacaactgaacatcaagctgacctctttcaaagcggttcatttctctccggtatccagcctgccggacacctccttcctgaaaggtccgtctccgcgtctgacctacctgtctgttgcgaatgcggatctgctgaaccgtacttggtcccgtggtggcaacgaacagtgcctgcgttacattgctaacctgatctcttgcttcccgagcgtctgcgttcgtgatgaaaaaggtaacccagtatcttggtctatcaccgaccagttcgcgaccatgtgtcatggctacaccctgcctgaacaccgtcgtaagggttactctcgtctggttgcgctgaccctggcgcgtaagctgcagtctcgtggttttccgtcccagggtaacgtcctggacgacaacactgcgtccatctccctgctgaagtccctgcatgccgaattcctgccgtgccgtttccaccgtctgattctgaccccagcgaccttctctggtctgccgcacctgtaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 10 - DNA, 693 bp, Pseudomonas putida
  misc_feature (1)..(693): alkL (an importer facilitating transport of hydrophobic acyl across cell membranes) gene
gtgagtttttctaattataaagtaatcgcgatgccggtgttggttgctaattttgttttgggggcggccactgcatgggcgaatgaaaattatccggcgaaatctgctggctataatcagggtgactgggtcgctagcttcaatttttctaaggtctatgtgggtgaggagcttggcgatctaaatgttggagggggggctttgccaaatgctgatgtaagtattggtaatgatacaacacttacgtttgatatcgcctattttgttagctcaaatatagcggtggatttttttgttggggtgccagctagggctaaatttcaaggtgagaaatcaatctcctcgctgggaagagtcagtgaagttgattacggccctgcaattctttcgcttcaatatcattacgatagctttgagcgactttatccatatgttggggttggtgttggtcgggtgctattttttgataaaaccgacggtgctttgagttcgtttgatattaaggataaatgggcgcctgcttttcaggttggccttagatatgaccttggtaactcatggatgctaaattcagatgtgcgttatattcctttcaaaacggacgtcacaggtactcttggcccggttcctgtttctactaaaattgaggttgatcctttcattctcagtcttggtgcgtcatatgttttctaa

SEQ ID NO: 11 - DNA, 153 bp, Artificial Sequence
  lacuv5 promoter
ggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcat

SEQ ID NO: 12 - DNA, 4876 bp, Artificial Sequence
  Vector pCDF[alkLmod1]
ggggaattgtgagcggataacaattcccctgtagaaataattttgtttaactttaataaggagatataccatgggcagcagccatcaccatcatcaccacagccaggatccttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggatccggccggcctgcagccccgcagggcctgtctcggtcgatcattcagcccggctcatagatatgcgggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcatgtgagtttttctaattataaagtaatcgcgatgccggtgttggttgctaattttgttttgggggcggccactgcatgggcgaatgaaaattatccggcgaaatctgctggctataatcagggtgactgggtcgctagcttcaatttttctaaggtctatgtgggtgaggagcttggcgatctaaatgttggagggggggctttgccaaatgctgatgtaagtattggtaatgatacaacacttacgtttgatatcgcctattttgttagctcaaatatagcggtggatttttttgttggggtgccagctagggctaaatttcaaggtgagaaatcaatctcctcgctgggaagagtcagtgaagttgattacggccctgcaattctttcgcttcaatatcattacgatagctttgagcgactttatccatatgttggggttggtgttggtcgggtgctattttttgataaaaccgacggtgctttgagttcgtttgatattaaggataaatgggcgcctgcttttcaggttggccttagatatgaccttggtaactcatggatgctaaattcagatgtgcgttatattcctttcaaaacggacgtcacaggtactcttggcccggttcctgtttctactaaaattgaggttgatcctttcattctcagtcttggtgcgtcatatgttttctaaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 13 - DNA, 7468 bp, Artificial Sequence
  Vector pCDF{Ptac}[hGLYAT2(co_Ec)-fadD_Ec] {Placuv5}[alkLmod1]
cgggatctcgacgctctcccttatgcgactcctgcgtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcattgtctcggtcgatcattcagcccggctcatagatatgcgggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcatgtgagtttttctaattataaagtaatcgcgatgccggtgttggttgctaattttgttttgggggcggccactgcatgggcgaatgaaaattatccggcgaaatctgctggctataatcagggtgactgggtcgctagcttcaatttttctaaggtctatgtgggtgaggagcttggcgatctaaatgttggagggggggctttgccaaatgctgatgtaagtattggtaatgatacaacacttacgtttgatatcgcctattttgttagctcaaatatagcggtggatttttttgttggggtgccagctagggctaaatttcaaggtgagaaatcaatctcctcgctgggaagagtcagtgaagttgattacggccctgcaattctttcgcttcaatatcattacgatagctttgagcgactttatccatatgttggggttggtgttggtcgggtgctattttttgataaaaccgacggtgctttgagttcgtttgatattaaggataaatgggcgcctgcttttcaggttggccttagatatgaccttggtaactcatggatgctaaattcagatgtgcgttatattcctttcaaaacggacgtcacaggtactcttggcccggttcctgtttctactaaaattgaggttgatcctttcattctcagtcttggtgcgtcatatgttttctaaggtaccctcgagtctggtggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgttaagcgttaagtctgagaggtgaagaattgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctgagcgttaagtcagtcgtcagacgccggttaatccggcgttttttttgacgcccactaaagagaaaacaatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtc

SEQ ID NO: 14 - DNA, 5367 bp, Artificial Sequence
  Vector pET-28b
ggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 15 - DNA, 6432 bp, Artificial Sequence
  Vector pET-28b{Ptac}[hGLYAT2(co_Ec)]
ggccgctctggtggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgcacaacagccaaaaactgcaaattctgtacaaatctctggaaaaatctattcctgaaagcattaaagtttatggtgcgatcttcaacatcaaggataaaaaccctttcaatatggaggtgctggtcgacgcgtggcctgattaccaaatcgtcatcacccgtccgcagaagcaagaaatgaaagatgaccaggaccactatactaacacctaccacatcttcactaaagcgccggacaaactggaagaagttctgagctactccaatgttatctcctgggaacaaacgctgcagattcaaggttgccaggaaggtctggatgaggcgattcgtaaggtcgctacctctaagtccgttcaagtcgattacatgaaaaccatcctgtttatcccggagctgccgaagaaacacaaaacctcctctaacgacaagatggagctgttcgaagtagacgatgacaacaaagagggtaacttttccaatatgttcctggacgcctcccacgccggtctggttaatgagcactgggcgttcggtaaaaacgaacgctccctgaagtacattgagcgttgtctgcaggattttctgggttttggtgtcctgggtccagagggtcaactggtctcttggatcgttatggaacagtcttgtgaactgcgtatgggttatactgtgccaaaataccgtcaccaaggtaatatgctgcaaatcggttatcatctggagaagtacctgagccagaaggaaatcccgttctatttccatgttgccgataataacgaaaagagcctgcaagccctgaacaatctgggcttcaagatctgcccttgcggttggcaccagtggaagtgcacgcctaaaaagtactgttaagcgttaagtctgagaggtgagagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgc

SEQ ID NO: 16 - DNA, 6418 bp, Artificial Sequence
  pET-28b{Ptac}[hGLYAT3(co_Ec)]
ggccgcccttcattttggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgctggttctgaattgctctactaaactgctgatcctggagaaaatgctgaaatcctgtttcccggaatctctgaaagtttatggtgcggtaatgaatatcaaccgtggtaatccgtttcaaaaagaagttgttctggactcttggccagatttcaaggcggttatcacccgtcgtcagcgtgaagcggaaaccgacaacctggaccactataccaacgcgtatgcggttttctataaagatgttcgcgcgtaccgtcagctgctggaagaatgcgatgttttcaactgggaccaggtttttcagatccaaggtctgcaatccgagctgtacgacgtttctaaggccgtggcgaactctaaacaactgaacatcaagctgacctctttcaaagcggttcatttctctccggtatccagcctgccggacacctccttcctgaaaggtccgtctccgcgtctgacctacctgtctgttgcgaatgcggatctgctgaaccgtacttggtcccgtggtggcaacgaacagtgcctgcgttacattgctaacctgatctcttgcttcccgagcgtctgcgttcgtgatgaaaaaggtaacccagtatcttggtctatcaccgaccagttcgcgaccatgtgtcatggctacaccctgcctgaacaccgtcgtaagggttactctcgtctggttgcgctgaccctggcgcgtaagctgcagtctcgtggttttccgtcccagggtaacgtcctggacgacaacactgcgtccatctccctgctgaagtccctgcatgccgaattcctgccgtgccgtttccaccgtctgattctgaccccagcgaccttctctggtctgccgcacctgtaagcgttaagtctgagaggtgagagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgc

SEQ ID NO: 17 - DNA, 4334 bp, Escherichia coli
  misc_feature (1)..(4334): pBMT-3_ccdAB
ctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccccctcaagtcaaaagcctccggtcggaggcttttgactttctgctatggaggtcaggtatgatttaaatggtcagtattgagcgatatctagagaattcgtcaacgaattcaagcttgatatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaag

SEQ ID NO: 18 - DNA, 5024 bp, Escherichia coli
  misc_feature (1)..(5024): pBMT-3_ccdAB_PT7-′tesA
caaaaaacccctcaagacccgtttagaggccccaaggggttatgctagttattgctcagcggtggcagcagcctaggttaattaagctgcgctagtagacgagtccatgtgctggcgttcaaatttcgcagcagcggtttctttaccagactcgaaaattcaggagtcgtggttgaccagcggctgcagttgcttggccatccagtcggcgatgaacggctgcgcgtcgcggttcgggtggatgccgtcgtcctgcatccactgcggcttcaggtagacttcctccatgaagaacggcagcagcggcacgtcgaattccttggccagcttcgggtagatcgcggagaaggcctcgttgtagcgacggccgtagttcgccggcaggcggatctgcatcagcagcggctcggcgttcgcggccttcacgtcctgcaggatctggcgcagcgtctgttcggtctgctgcggctggaagccgcgcaggccgtcgttgccgcccagctcgaccagcacccagcgcggctggtgctgcttcagcagcgccggcaggcgggccaggccctgctggctcgtgtcgccgctgatcgaggcgttgaccaccgaggtcttggactgccacttgtcgttcagcagcgccggccacgccgccgacgccgacatgcggtagcccgccgacagggagtcgcccaggatcagcagggtgtccgccgccatggtatatctccttattaaagttaaacaaaattatttctacaggggaattgttatccgctcacaattcccctatagtgagtcgtattaatttcctaatgcaggagtcgatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaa

SEQ ID NO: 19 - DNA, 8268 bp, Escherichia coli
  misc_feature (1)..(8268): pBMT-3_ccdAB_PT7-nphT7-hbd-crt-ter
ccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaaaaatcccttatgcgactcctgcattaggaaattaatacgactcactataggggaattgtgagcggataacaattcccctgtagaaataattttgtttaactttaataaggagatataccatgacggatgtccgctttcgtatcattggcacgggcgcttacgtcccggaacgcattgtcagcaacgacgaagtcggcgcaccggctggtgtggatgacgattggattacgcgtaaaaccggtatccgccagcgtcgctgggcggccgacgatcaagcaacgagcgatctggctaccgcagctggtcgtgcagcactgaaagcagctggcattacgccggaacagctgaccgtcatcgcagtggctacctctacgccggaccgtccgcagccgccgaccgcggcctatgttcaacatcacctgggtgcaaccggcacggcagcttttgatgttaacgctgtgtgcagcggtacggttttcgcgctgagctctgttgccggcaccctggtctatcgtggcggttacgcactggtcattggtgctgatctgtactcacgtatcctgaatccggcggaccgcaaaaccgtggttctgttcggcgatggtgcgggcgcgatggtgctgggcccgaccagcaccggcaccggtccgattgtgcgtcgcgttgcactgcatacgtttggcggtctgaccgatctgatccgtgttccggccggcggttcccgccagccgctggacaccgatggtctggacgcaggcctgcaatattttgcgatggatggccgcgaagtccgtcgcttcgtgaccgaacatctgccgcagctgattaaaggttttctgcacgaagcgggcgtggatgcggcggatattagccatttcgtgccgcaccaagccaacggtgtgatgctggacgaagtttttggcgaactgcatctgccgcgtgcaaccatgcaccgtacggtggaaacctacggtaatacgggcgcagctagtattccgatcacgatggatgcggccgttcgtgcaggttccttccgtccgggcgaactggttctgctggcgggctttggcggcggtatggcggcttcgtttgctctgattgaatggtgacctaatgcaggctgcaggcggatacgaggaggaataaaccatgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataagtttacaggatctgcagggaggaggaaatcatggagttgaacaacgttattctggagaaagaaggcaaggtggcggttgtcaccattaaccgtccaaaggccctgaacgctctgaactcggataccctgaaagagatggattacgttattggcgagattgagaatgacagcgaagtgctggctgtgattctgaccggtgcgggtgagaagagctttgtcgcgggtgcggacatcagcgagatgaaagaaatgaacaccatcgaaggccgtaagttcggtattctgggcaacaaggtgtttcgtcgtctggaactgctggagaaacctgtcattgctgccgtgaacggtttcgcgctgggcggtggttgcgagatcgctatgagctgcgatattcgtatcgcatcgtccaacgcacgctttggtcaaccggaggtcggtctgggtatcactccgggtttcggcggtacgcaacgtctgagccgcctggttggcatgggcatggcgaaacagttgattttcacggcacagaacattaaggcggatgaggcgctgcgtattggtctggtgaataaggtcgttgagccaagcgaactgatgaataccgcgaaagaaattgcgaacaagatcgttagcaatgccccggtggccgttaagctgtcgaaacaggcaatcaaccgtggcatgcagtgtgacatcgacaccgccctggcgtttgagagcgaggcgtttggtgagtgcttctccaccgaggaccaaaaggatgcgatgaccgcgttcattgagaaacgcaagatcgagggtttcaagaatcgttaatagaggaggataggaggttttcatatgattgtgaaaccgatggtccgtaataatatctgtctgaatgctcacccgcagggctgtaaaaaaggcgtggaagatcaaattgaatataccaaaaaacgtattacggcagaagtgaaagccggcgcaaaagctccgaaaaacgtgctggttctgggttgcagcaatggctatggtctggcttctcgcattaccgcggcctttggctacggtgcagctacgatcggcgttagtttcgaaaaagcaggttccgaaaccaaatatggcacgccgggttggtacaacaatctggcttttgatgaagcggccaaacgtgaaggcctgtatagtgtcaccattgatggtgacgcgttctccgatgaaattaaagcacaggtgatcgaagaagcgaagaaaaaaggcattaaatttgacctgatcgtttacagcctggcatctccggtccgtaccgatccggacacgggtatcatgcataaatctgtgctgaaaccgtttggcaaaaccttcacgggtaaaaccgttgatccgttcacgggcgaactgaaagaaattagcgcggaaccggccaacgatgaagaagcagctgcgaccgtcaaagtgatgggcggtgaagactgggaacgttggatcaaacagctgagtaaagaaggcctgctggaagaaggttgcattaccctggcgtattcctacatcggcccggaagcaacccaagctctgtatcgcaaaggcacgattggtaaagcgaaagaacatctggaagcgaccgcccaccgtctgaacaaagaaaatccgtcaatccgcgccttcgtttcggtcaataaaggtctggttacccgtgcatcagctgtgattccggttatcccgctgtacctggcatcgctgtttaaagtcatgaaagaaaaaggcaaccatgaaggttgtattgaacagatcacccgcctgtatgccgaacgtctgtaccgcaaagatggtacgattccggtggacgaagaaaatcgtattcgcatcgatgactgggaactggaagaagatgtccaaaaagccgtgagcgccctgatggaaaaagttaccggcgaaaacgcggaatctctgacggatctggccggttatcgtcacgactttctggcgagtaatggttttgatgttgaaggcattaactacgaagctgaagtggaacgctttgatcgcatttgatctagagaattcgtcaacgaattcaagcttgatatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctc

SEQ ID NO: 20 - DNA, 8959 bp, Escherichia coli
  misc_feature (1)..(8959): pBMT-3_ccdAB_PT7-?tesA_PT7-nphT7-hbd-crt-ter
ccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtacccggaattgccagctggggcgccctctggtaaggttgggaagccctgcaaagtaaactggatggctttcttgccgccaaggatctgatggcgcaggggatcaagctctgatcaagagacaggatgaggatcgtttcgatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagcgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatcctagacgaattctctagtagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatcttctcgacgctctcccttatgcgactcctgcattaggaaattaatacgactcactataggggaattgtgagcggataacaattcccctgtagaaataattttgtttaactttaataaggagatataccatggcggcggacaccctgctgatcctgggcgactccctgtcggcgggctaccgcatgtcggcgtcggcggcgtggccggcgctgctgaacgacaagtggcagtccaagacctcggtggtcaacgcctcgatcagcggcgacacgagccagcagggcctggcccgcctgccggcgctgctgaagcagcaccagccgcgctgggtgctggtcgagctgggcggcaacgacggcctgcgcggcttccagccgcagcagaccgaacagacgctgcgccagatcctgcaggacgtgaaggccgcgaacgccgagccgctgctgatgcagatccgcctgccggcgaactacggccgtcgctacaacgaggccttctccgcgatctacccgaagctggccaaggaattcgacgtgccgctgctgccgttcttcatggaggaagtctacctgaagccgcagtggatgcaggacgacggcatccacccgaaccgcgacgcgcagccgttcatcgccgactggatggccaagcaactgcagccgctggtcaaccacgactcctgaatttaaatcccttatgcgactcctgcattaggaaattaatacgactcactataggggaattgtgagcggataacaattcccctgtagaaataattttgtttaactttaataaggagatataccatgacggatgtccgctttcgtatcattggcacgggcgcttacgtcccggaacgcattgtcagcaacgacgaagtcggcgcaccggctggtgtggatgacgattggattacgcgtaaaaccggtatccgccagcgtcgctgggcggccgacgatcaagcaacgagcgatctggctaccgcagctggtcgtgcagcactgaaagcagctggcattacgccggaacagctgaccgtcatcgcagtggctacctctacgccggaccgtccgcagccgccgaccgcggcctatgttcaacatcacctgggtgcaaccggcacggcagcttttgatgttaacgctgtgtgcagcggtacggttttcgcgctgagctctgttgccggcaccctggtctatcgtggcggttacgcactggtcattggtgctgatctgtactcacgtatcctgaatccggcggaccgcaaaaccgtggttctgttcggcgatggtgcgggcgcgatggtgctgggcccgaccagcaccggcaccggtccgattgtgcgtcgcgttgcactgcatacgtttggcggtctgaccgatctgatccgtgttccggccggcggttcccgccagccgctggacaccgatggtctggacgcaggcctgcaatattttgcgatggatggccgcgaagtccgtcgcttcgtgaccgaacatctgccgcagctgattaaaggttttctgcacgaagcgggcgtggatgcggcggatattagccatttcgtgccgcaccaagccaacggtgtgatgctggacgaagtttttggcgaactgcatctgccgcgtgcaaccatgcaccgtacggtggaaacctacggtaatacgggcgcagctagtattccgatcacgatggatgcggccgttcgtgcaggttccttccgtccgggcgaactggttctgctggcgggctttggcggcggtatggcggcttcgtttgctctgattgaatggtgacctaatgcaggctgcaggcggatacgaggaggaataaaccatgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataagtttacaggatctgcagggaggaggaaatcatggagttgaacaacgttattctggagaaagaaggcaaggtggcggttgtcaccattaaccgtccaaaggccctgaacgctctgaactcggataccctgaaagagatggattacgttattggcgagattgagaatgacagcgaagtgctggctgtgattctgaccggtgcgggtgagaagagctttgtcgcgggtgcggacatcagcgagatgaaagaaatgaacaccatcgaaggccgtaagttcggtattctgggcaacaaggtgtttcgtcgtctggaactgctggagaaacctgtcattgctgccgtgaacggtttcgcgctgggcggtggttgcgagatcgctatgagctgcgatattcgtatcgcatcgtccaacgcacgctttggtcaaccggaggtcggtctgggtatcactccgggtttcggcggtacgcaacgtctgagccgcctggttggcatgggcatggcgaaacagttgattttcacggcacagaacattaaggcggatgaggcgctgcgtattggtctggtgaataaggtcgttgagccaagcgaactgatgaataccgcgaaagaaattgcgaacaagatcgttagcaatgccccggtggccgttaagctgtcgaaacaggcaatcaaccgtggcatgcagtgtgacatcgacaccgccctggcgtttgagagcgaggcgtttggtgagtgcttctccaccgaggaccaaaaggatgcgatgaccgcgttcattgagaaacgcaagatcgagggtttcaagaatcgttaatagaggaggataggaggttttcatatgattgtgaaaccgatggtccgtaataatatctgtctgaatgctcacccgcagggctgtaaaaaaggcgtggaagatcaaattgaatataccaaaaaacgtattacggcagaagtgaaagccggcgcaaaagctccgaaaaacgtgctggttctgggttgcagcaatggctatggtctggcttctcgcattaccgcggcctttggctacggtgcagctacgatcggcgttagtttcgaaaaagcaggttccgaaaccaaatatggcacgccgggttggtacaacaatctggcttttgatgaagcggccaaacgtgaaggcctgtatagtgtcaccattgatggtgacgcgttctccgatgaaattaaagcacaggtgatcgaagaagcgaagaaaaaaggcattaaatttgacctgatcgtttacagcctggcatctccggtccgtaccgatccggacacgggtatcatgcataaatctgtgctgaaaccgtttggcaaaaccttcacgggtaaaaccgttgatccgttcacgggcgaactgaaagaaattagcgcggaaccggccaacgatgaagaagcagctgcgaccgtcaaagtgatgggcggtgaagactgggaacgttggatcaaacagctgagtaaagaaggcctgctggaagaaggttgcattaccctggcgtattcctacatcggcccggaagcaacccaagctctgtatcgcaaaggcacgattggtaaagcgaaagaacatctggaagcgaccgcccaccgtctgaacaaagaaaatccgtcaatccgcgccttcgtttcggtcaataaaggtctggttacccgtgcatcagctgtgattccggttatcccgctgtacctggcatcgctgtttaaagtcatgaaagaaaaaggcaaccatgaaggttgtattgaacagatcacccgcctgtatgccgaacgtctgtaccgcaaagatggtacgattccggtggacgaagaaaatcgtattcgcatcgatgactgggaactggaagaagatgtccaaaaagccgtgagcgccctgatggaaaaagttaccggcgaaaacgcggaatctctgacggatctggccggttatcgtcacgactttctggcgagtaatggttttgatgttgaaggcattaactacgaagctgaagtggaacgctttgatcgcatttgatctagagaattcgtcaacgaattcaagcttgatatcattcaggacgagcctcagactccagcgtaactggactgaaaacaaactaaagcgcccttgtggcgctttagttttgttccgctcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctc

SEQ ID NO: 21 - DNA, 5367 bp, Escherichia coli; identical to SEQ ID NO: 14
  misc_feature (1)..(5367): pET-28b(empty vector)

SEQ ID NO: 22 - DNA, 43 bp, Artificial Sequence
  oligonucleotide o-MO-40 for gcvTHP amplification
ccggggatcgcggccgcggaccgaacttatccgccaggcaatg

SEQ ID NO: 23 - DNA, 51 bp, Artificial Sequence
  oligonucleotide o-MO-41 for gcvTHP amplification
ttactggtattcgctaatcggctcgagcatcttgtcctcattgaataagcg

SEQ ID NO: 24 - DNA, 30 bp, Artificial Sequence
  oligonucleotide o-MO-42 for gcvTHP amplification
ctcgagccgattagcgaataccagtaattc

SEQ ID NO: 25 - DNA, 41 bp, Artificial Sequence
  oligonucleotide o-MO-43 for gcvTHP amplification
ggatcgcggccgctctagagttcatcgtgctgatcgattgc

SEQ ID NO: 26 - DNA, 553 bp, Artificial Sequence
  PCR product 1 from example 12, 1553bp
ccggggatcgcggccgcggaccgaacttatccgccaggcaatgggattaaacgatttgcctgaatggctgcgttaaaaatttctcctctgttgtttatttgatacccatcacactttcatctcccggttttttcgccgggagattttcctcatttgaaataaactaatttcacctccgttttcgcattatattttctaatgccattattttttgatttagtgttttttgacatttttttagctcttaatattgtcttattcaaattgactttctcatcacatcatctttgtatagaaactggtgtattttttggttttttattctgtcgcgatttttgcattttttaaccataagctaatgtgatgatcaattttaccttatggttaacagtctgtttcggtggtaagttcaggcaaaagagaacgattgcgttggggaccgggagtggctccgatgctgggtttcgtggtgataatttcaccatgaaaaagttgtcagccccgcttattcaatgaggacaagatgctcgagccgattagcgaataccagtaa

SEQ ID NO: 27 - DNA, 547 bp, Artificial Sequence
  PCR product 2 from example 12, 1553bp
ctcgagccgattagcgaataccagtaattcactgattcgactattttctaaaggcgcttcggcgcctttttagtcagatgacaaagtacaaaagtgctcagacagtcccctcgcccctttggggagagggttagggtgaggggaacaggccggcactggcgcgaatatttaccctcaccccggccctctccctgaaagggcgagggggaaaagcgtgccaacattgaagattgagccagtttgttagcaatctcaaagatacgtcaacgaattaatttttctcggaaaaacaaatggctatagcacttgtgactggtggcagtcgcggcatcgggcgggcaactgcattactgttggcgcaagaagggtatacggtggcggttaattatcagcaaaacctccacgcggcgcaggaagtgatgaacttaataacgcaagccggtggcaaagcattcgtgctccaggcggatatcagcgacgaaaaccaggtcgttgcgatgtttacagcaatcgatcagcacgatgaactctagagcggccgcgatcc

SEQ ID NO: 28 - DNA, 5667 bp, Artificial Sequence
  vector pKO3
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccggatcctctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 29 - DNA, 6693 bp, Artificial Sequence
  knock-out vector pKO3 delta gcvTHP
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccgaacttatccgccaggcaatgggattaaacgatttgcctgaatggctgcgttaaaaatttctcctctgttgtttatttgatacccatcacactttcatctcccggttttttcgccgggagattttcctcatttgaaataaactaatttcacctccgttttcgcattatattttctaatgccattattttttgatttagtgttttttgacatttttttagctcttaatattgtcttattcaaattgactttctcatcacatcatctttgtatagaaactggtgtattttttggttttttattctgtcgcgatttttgcattttttaaccataagctaatgtgatgatcaattttaccttatggttaacagtctgtttcggtggtaagttcaggcaaaagagaacgattgcgttggggaccgggagtggctccgatgctgggtttcgtggtgataatttcaccatgaaaaagttgtcagccccgcttattcaatgaggacaagatgctcgagccgattagcgaataccagtaattcactgattcgactattttctaaaggcgcttcggcgcctttttagtcagatgacaaagtacaaaagtgctcagacagtcccctcgcccctttggggagagggttagggtgaggggaacaggccggcactggcgcgaatatttaccctcaccccggccctctccctgaaagggcgagggggaaaagcgtgccaacattgaagattgagccagtttgttagcaatctcaaagatacgtcaacgaattaatttttctcggaaaaacaaatggctatagcacttgtgactggtggcagtcgcggcatcgggcgggcaactgcattactgttggcgcaagaagggtatacggtggcggttaattatcagcaaaacctccacgcggcgcaggaagtgatgaacttaataacgcaagccggtggcaaagcattcgtgctccaggcggatatcagcgacgaaaaccaggtcgttgcgatgtttacagcaatcgatcagcacgatgaactctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 30 - DNA, 30 bp, Artificial Sequence
  DNA sequence after deletion of gcvTHP
atgctcgagccgattagcgaataccagtaa

SEQ ID NO: 31 - DNA, 38 bp, Artificial Sequence
  oligonucleotide o-MO-44 for amplifying the upstream region of glyA
gggatcgcggccgcggaccgagggcttcacgttgatcg

SEQ ID NO: 32 - DNA, 45 bp, Artificial Sequence
  oligonucleotide o-MO-45 for amplifying the upstream region of glyA
tatgcgtaaaccgggtaacgctcgagcatccgcatctcctgactc

SEQ ID NO: 33 - DNA, 22 bp, Artificial Sequence
  oligonucleotide o-MO-46 for amplifying the downstream region of glyA
cgttacccggtttacgcataag

SEQ ID NO: 34 - DNA, 39 bp, Artificial Sequence
  oligonucleotide o-MO-47 for amplifying the downstream region of glyA
ggatcgcggccgctctagagatcaccagctgggttgatc

SEQ ID NO: 35 - DNA, 546 bp, Artificial Sequence
  PCR product 1 from Example 14
gggatcgcggccgcggaccgagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcggatgctcgagcgttacccggtttacgcata

SEQ ID NO: 36 - DNA, 520 bp, Artificial Sequence
  PCR product 2 from Example 14
cgttacccggtttacgcataagcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatctctagagcggccgcgatcc

SEQ ID NO: 37 - DNA, 26 bp, Artificial Sequence
  oligonucleotide o-MO-52
actgaggatccgggatcgcggccgcg

SEQ ID NO: 38 - DNA, 31 bp, Artificial Sequence
  oligonucleotide o-MO-53
actgaggatccggatcgcggccgctctagag

SEQ ID NO: 39 - DNA, 1052 bp, Artificial Sequence
  PCR product 3 from Example 14
gatccgggatcgcggccgcggaccgagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcggatgctcgagcgttacccggtttacgcataagcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatctctagagcggccgcgatccg

SEQ ID NO: 40 - DNA, 6719 bp, Artificial Sequence
  pKO3 delta glyA
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccggatccgggatcgcggccgcggaccgagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcggatgctcgagcgttacccggtttacgcataagcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatctctagagcggccgcgatccggatcctctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 41 - DNA, 30 bp, Artificial Sequence
  DNA sequence after deletion of glyA
atgctcgagcgttacccggtttacgcataa

SEQ ID NO: 42 - DNA, 39 bp, Artificial Sequence
  ltaE-UP_fw
ggatcgcggccgcggaccgcaggcgacagagccagaacg

SEQ ID NO: 43 - DNA, 47 bp, Artificial Sequence
  ltAE-UP-XhoI_rev
ctctccttaacgcgccaggaactcgagcatggcatgtccttattatg

SEQ ID NO: 44 - DNA, 21 bp, Artificial Sequence
  ltaE-DOWN_fw
ttcctggcgcgttaaggagag

SEQ ID NO: 45 - DNA, 40 bp, Artificial Sequence
  ltaE-DOWN_rev
ggatcgcggccgctctagagtagaccatatcgcgcatgac

SEQ ID NO: 46 - DNA, 540 bp, Artificial Sequence
  PCR product 4 from Example 15
ggatcgcggccgcggaccgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaaaaagggtggcatttcccgtcataataaggacatgccatgctcgagttcctggcgcg

SEQ ID NO: 47 - DNA, 536 bp, Artificial Sequence
  PCR product 5 from Example 15
ttcctggcgcgttaaggagagagacgtgccgcaacgcattttagttctcggtgccagtggctacattggtcagcatctggtgcgcacactcagccagcaagggcatcagatcctggcggcggcacgtcatgtcgacaggcttgcaaagctgcaactggcaaacgtcagttgccataaagtcgatctcagctggccggataacctaccggccctgttgcaggatatcgatacggtctattttctggtgcacagcatgggcgaaggcggcgattttatcgctcaggagcgccaggtggctctcaacgtccgcgatgcgctacgtgaagtaccagttaagcaattaatctttcttagttcgttgcaggccccgccacatgagcagtcggatcatctgcgtgctcgtcaggctacggcggacattctgcgtgaagcgaatgtacccgtgaccgaactgcgggccggaattatcgttggcgcaggttccgcggcgttcgaagtcatgcgcgatatggtctactctagagcggccgcgatcc

SEQ ID NO: 48 - DNA, 26 bp, Artificial Sequence
  oligonucleotide o-MO-54 from Example 15
actgaggatccggatcgcggccgcgg

SEQ ID NO: 49 - DNA, 29 bp, Artificial Sequence
  oligoneucleotide o-MO-55 from Example 15
actgaggatccggatcgcggccgctctag

SEQ ID NO: 50 - DNA, 1071 bp, Artificial Sequence
  PCR product 6 from Example 15
gatccggatcgcggccgcggaccgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaaaaagggtggcatttcccgtcataataaggacatgccatgctcgagttcctggcgcgttaaggagagagacgtgccgcaacgcattttagttctcggtgccagtggctacattggtcagcatctggtgcgcacactcagccagcaagggcatcagatcctggcggcggcacgtcatgtcgacaggcttgcaaagctgcaactggcaaacgtcagttgccataaagtcgatctcagctggccggataacctaccggccctgttgcaggatatcgatacggtctattttctggtgcacagcatgggcgaaggcggcgattttatcgctcaggagcgccaggtggctctcaacgtccgcgatgcgctacgtgaagtaccagttaagcaattaatctttcttagttcgttgcaggccccgccacatgagcagtcggatcatctgcgtgctcgtcaggctacggcggacattctgcgtgaagcgaatgtacccgtgaccgaactgcgggccggaattatcgttggcgcaggttccgcggcgttcgaagtcatgcgcgatatggtctactctagagcggccgcgatccg

SEQ ID NO: 51 - DNA, 6738 bp, Artificial Sequence
  pKO3 delta ltaE
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgcggccgcggaccggatccggatcgcggccgcggaccgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaaaaagggtggcatttcccgtcataataaggacatgccatgctcgagttcctggcgcgttaaggagagagacgtgccgcaacgcattttagttctcggtgccagtggctacattggtcagcatctggtgcgcacactcagccagcaagggcatcagatcctggcggcggcacgtcatgtcgacaggcttgcaaagctgcaactggcaaacgtcagttgccataaagtcgatctcagctggccggataacctaccggccctgttgcaggatatcgatacggtctattttctggtgcacagcatgggcgaaggcggcgattttatcgctcaggagcgccaggtggctctcaacgtccgcgatgcgctacgtgaagtaccagttaagcaattaatctttcttagttcgttgcaggccccgccacatgagcagtcggatcatctgcgtgctcgtcaggctacggcggacattctgcgtgaagcgaatgtacccgtgaccgaactgcgggccggaattatcgttggcgcaggttccgcggcgttcgaagtcatgcgcgatatggtctactctagagcggccgcgatccggatcctctagagcggccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 52 - DNA, 24 bp, Artificial Sequence
  DNA sequence after deletion of ltaE
atgctcgagttcctggcgcgttaa

SEQ ID NO: 53 - protein, 294 aa, Nomocharis pardanthina
MLVLHNAQKLQILYKSLEKSIPESIKVYGAIFNIKDKNPFNMEVLVDAWPDYQIVITRPQKQEMKDDQDHYTNTYHIFTKAPDKLEEVLSYSSVINWEQTLQIQGCQEGLDEAIRKAATSKSVQVDYMKTMLFIPELPKKHKTSSNEKMELFKVDDDNKEGNFSNMFLDASHAGLVNEHWAFGKNERSLKYIERCLQDFLGFGVLGPEGQLVSWIVMEQSCELRMGYTVPKYRHQGNMLQIGYHFEKYLSQKEIPFYFHVADNNEKSLQALKNLGFKICPCGWHQWKCTPKKYC

SEQ ID NO: 54 - protein, 297 aa, Saimiri boliviensis
MFVLHDTQILQTLYKSLEKRNPESIKVYGAIFNIKNKNPFNMEVLVDAWPDYQTVIIRPKKQEMKDDQDHYTNTYHIFTKAPDKLEEILSCSEVINWEQTFQIQGCQEGLNEAIRKVAASKTVQVDYVKTVLFKPELLKEHKTSSNDKMELFEMHKVDDNNKKRNFSSMFLDASHAGLINEHWAFGKNERSLKYTERCLQDFLGFGVLGPEGELISWVVMEQSCELRMGYTIPKYRNQRHMSQILCYLEKYLSQKEIPFYIHVADYNVAGLQIMKNMGYMICPCGWHQWKCTPKKYC

SEQ ID NO: 55 - protein, 298 aa, Felis catus
MFVLHESQKLHILYESLEKSIPESLKVYGAVFNIKNKNPFNMEVLVDAWPDYQIVITRPQREEMKDDLDHYTNTYQIFTKAPDKLEEVLAQPQLINWDQVFQIQGCQESLQEAIRKISASKSVRVDYVKTIRIMTEMPVKPNDDQVDVMESFKMPKVDDDTKKGSFQNISLDPSHAGLVNEQWAFGKNDRSLKYIERCLQNFPGFGVLGPEGRLISWIVMEQSCEMRMGYTVPEYRKHGYMKKTSVHLMNYVIQKKVPCYFHVSEHKEEYNQALRDLKFKAASCGWHQWKCSPEKYVD

SEQ ID NO: 56 - protein, 299 aa, Bos taurus
MFVLREPQQLQILYESLEESIPESLKVYGTLFHIRNKNPFNLEVLVDAWPEYQTVVIRPQKEEMKDDLDYYTNTYHIFTKAPDKLEEVLACPQVINWEQAFQIQGCQESLGEAIQKVAASKSVQVDYLRTVLFVFEKPTLEASSGDKMDLMKLLKIPKVLEDKRKETFQNIFLDVSHARLVNEHWELGKNEKSLKYVERCLQNFAGFGVLSSEGKPISWFLTEQSCEIRMGYTFPKYRGQGHMWQMGCHSIAYFFSKKIPFYIHAAEDKEKTQQTLLSFGFKNVLCGWHQWKCTPKKYC

SEQ ID NO: 57 - protein, 296 aa, Mus musculus
MIVPLQGAQMLQMLEKSLRKYLPESLKVYGTVYHMIHGNPFNLKALVDKWPDFNTVVVRPQEQEMTDDLDFYINTYQVYSKDPQNCQEFLESSEVINWKQHLQIQSSQSHLNKTIQNLASIQSFQIKHSENILYVSSETIKKLFPSLLDTKNLSTGSGKPKAIDQDKFKLSSLDVVHAALVNKFWLFGGNERSQRFIERCIKNFPSSCVLGPEGTPASWTLMDQTGEMRMGGTMPEYRLQGLVSFVVHSQDQIMTKRGYPVYSHTEKSNIAMQKMSYTLQHLPMPCAWNQWKCMPM

(19) United States
(12) Patent Application Publication
(10) Pub. No.: US 2016/0272950 A1
(43) Pub. Date: Sep. 22, 2016

(54) METHOD FOR PRODUCING ACYL AMINO ACIDS

(71) Applicants: Jasmin CORTHALS, Bochum (DE); Katrin GRAMMANN, Oer-Erkenschwick (DE); Thomas HAAS, Muenster (DE); Maik OLFERT, Recklinghausen (DE); Nicole POTGRAVE, Dorsten (DE); Liv REINECKE, Essen (DE); Steffen SCHAFFER, Herten (DE); Jan WOLTER, Duesseldorf (DE)

(72) Inventors: Jasmin CORTHALS, Bochum (DE); Katrin GRAMMANN, Oer-Erkenschwick (DE); Thomas HAAS, Muenster (DE); Maik OLFERT, Recklinghausen (DE); Nicole POTGRAVE, Dorsten (DE); Liv REINECKE, Essen (DE); Steffen SCHAFFER, Herten (DE); Jan WOLTER, Duesseldorf (DE)

(73) Assignee: EVONIK DEGUSSA GMBH, Essen (DE)

(21) Appl. No.: 14/915,012

(22) PCT Filed: Aug. 25, 2014
(86) PCT No.: PCT/EP14/67990

§ 371 Date: Feb. 26, 2016

(30) Foreign Application Priority Data
Aug. 27, 2013 (EP) 13181841.1
May 23, 2014 (EP) 14169650.0

(51) Int. Cl. C12N 9/10; C12P 13/04

(52) U.S. Cl. CPC C12N 9/1029; C12P 13/04; C12Y 203/01086; C12Y 203/01065

(57) ABSTRACT

The present invention relates to a cell expressing an amino acid-N-acyl-transferase, which is preferably recombinant, and an acyl-CoA synthetase, which is preferably recombinant, wherein the cell has a reduced fatty acid degradation capacity, a method for producing acyl amino acids, comprising the step contacting an amino acid and an acyl CoA in the presence of an amino acid-N-acyl-transferase, which is preferably isolated and/or recombinant, wherein the amino acid-N-acyl-transferase is preferably a human amino acid-N-acyl-transferase, or culturing the cell and a reaction mixture comprising an amino acid-N-acyl-transferase, which is preferably isolated and/or recombinant, an acyl-CoA synthetase, which is preferably isolated and/or recombinant, an amino acid and either a fatty acid-CoA or a fatty acid and an acyl-CoA-synthase.

The present invention relates to a cell expressing an amino acid-N-acyl-transferase, which is preferably recombinant, and an acyl-CoA synthetase, which is preferably recombinant, wherein the cell has a reduced fatty acid degradation capacity, a method for producing acyl amino acids, comprising the step contacting an amino acid and fatty acid-CoA in the presence of an amino acid-N-acyl-transferase, which is preferably isolated and/or recombinant, wherein the amino acid-N-acyl-transferase is preferably a human amino acid-N-acyl-transferase, or culturing the cell, and a reaction mixture comprising an amino acid-N-acyl-transferase, which is preferably isolated and/or recombinant, an acyl-CoA synthetase, which is preferably isolated and/or recombinant, an amino acid and either a fatty acid-CoA or a fatty acid and an acyl-CoA-synthase.

Acyl amino acids are a class of surface-active agents with a variety of uses, for example as detergents for washing purposes, emulsifiers in food products and as essential ingredients in various personal care products such as shampoos, soaps, moisturizing agents and the like. In addition to having both hydrophobic and hydrophilic regions, a prerequisite for use as a surfactant, the compounds (surfactants) are made of naturally occurring molecules, more specifically amino acids and fatty acids, which are not only non-hazardous and environmentally acceptable but may be readily produced at a large scale using inexpensive biological raw materials. In pharmacological research, acyl amino acids are used as neuromodulators and probes for new drug targets.

Acyl amino acids have been isolated from a multitude of biological sources and are believed to have a range of functions, for example as signalling molecules in mammalian tissues (Tan, B., O'Dell, D. K., Yu, Y. W., Monn, M. F., Hughes, H. V., Burstein, S., Walker, J. M. (2010), Identification of endogenous acyl amino acids based on a targeted lipidomics approach, J. Lipid Res. 51(1), 112-119)), as building blocks for antibiotics in bacterial cultures (Clardy, J., and Brady, S. F. (2007), Cyclic AMP directly activates NasP, an N-acyl amino acid antibiotic biosynthetic enzyme cloned from an uncultured beta-proteobacterium, J. Bacteriol. 189(17), 6487-6489) or as compounds involved in bacterial protein sorting (Craig, J. W., Cherry, M. A., Brady, S. F. (2011), Long-chain N-acyl amino acid synthases are linked to the putative PEP-CTERM/exosortase protein-sorting system in Gram-negative bacteria, J. Bacteriol. 193(20), 5707-5715).

Traditionally acyl amino acids have been produced at an industrial scale starting with materials derived from petrochemicals. More specifically, activated fatty acids provided in the form of acid chlorides may be used to acylate amino acids in an aqueous alkaline medium as described in GB 1 483 500. Shortcomings of such approaches include the need to add hazardous chemicals such as sulphuric acid or anhydrides thereof. Other synthetic approaches are associated with the accumulation of by-products such as chloride salts which have undesirable effects on surfactancy.

A range of biotechnological routes towards production of acyl amino acids has been described. However, none of them is adequate for the commercial large-scale production of acyl amino acids owing to low yields, insufficient purities and the need for multi-step purification procedures. In particular, only a small proportion of the carbon substrates fed to biotechnologically useful organisms is actually converted to the sought-after product, whilst much of it is consumed by reactions of the primary metabolism.

Another problem associated with biotechnological routes is the fact that a mixture of products is obtained and thus the composition is difficult to control. More specifically, a range of fatty acids may be converted to acyl amino acids, even though production of a single adduct may be desirable. Since the mixture comprises compounds highly related in terms of chemical structure, purifying or at least enriching a single component in an efficient and straightforward manner is usually beyond technical feasibility.

These problems may be solved by the subject matter of the attached claims. In particular, the present invention may provide an efficient biotechnological route towards acyl amino acids. In particular, the yield and purity of the product of the present invention, in terms of catalysts or unwanted by-products, may be improved compared to the processes in the state of the art.

The present invention also provides a method for making acyl amino acids, wherein the spectrum of fatty acids converted to acyl amino acids is broader than the processes in the state of the art. In particular, the method of the present invention may be suitable for converting short and unsaturated fatty acids to acyl amino acids.

The present invention may also provide a biotechnological method for making acyl amino acids, wherein the length of the acyl residue in the acyl amino acid product may be controlled, preferably such that lauryl is enriched or prevalent.

In a first aspect, the present invention provides a cell expressing an amino acid-N-acyl-transferase, which may be preferably recombinant, and an acyl-CoA synthetase, which may be preferably recombinant, wherein the cell has a reduced fatty acid degradation capacity. In particular, the cell may compared to the wild type cell, have increased expression of amino acid-N-acyl-transferase and/or acyl-CoA synthetase.

In a first embodiment of the first aspect, the fatty acid degradation capacity of said cell may be reduced owing to a decrease in activity, compared to the wild type cell, of at least one enzyme selected from the group consisting of acyl-CoA dehydrogenase, 2,4-dienoyl-CoA reductase, enoyl-CoA hydratase and 3-ketoacyl-CoA thiolase, preferably acyl-CoA dehydrogenase.

In a second embodiment, which is also an embodiment of the first embodiment, wherein the amino acid-N-acyl-transferase may be a human amino acid-N-acyl-transferase, preferably SEQ ID NO: 4, SEQ ID NO: 5 or a variant thereof. In particular, the amino acid-N-acyl-transferase may have a polypeptide sequence comprising SEQ ID NO: 4, SEQ ID NO: 5 or a variant thereof.

In a third embodiment, which is also an embodiment of the first to second embodiment, the cell may be a bacterial cell, preferably an enterobacterial cell, more preferably E. coli.

In a fourth embodiment, which is also an embodiment of the first to third embodiments, the cell may be capable of making proteinogenic amino acids and/or fatty acids.

In a fifth embodiment, which is also an embodiment of the first to fourth embodiments, the cell expresses an acyl-CoA thioesterase, which is preferably recombinant and is more preferably SEQ ID NO: 1 or a variant thereof.

In a sixth embodiment, which is also an embodiment of the first to fifth embodiments, the acyl-CoA synthetase may be SEQ ID NO: 6 or a variant thereof.

In a second aspect the present invention provides a method for producing acyl amino acids, comprising the step of
b) contacting an amino acid and an acyl CoA in the presence of an amino acid-N-acyl-transferase, which may be preferably isolated and/or recombinant,
wherein the amino acid-N-acyl-transferase may be preferably a human amino acid-N-acyl-transferase, more preferably SEQ ID NO 4, SEQ ID NO 5 or a variant thereof, and wherein the acyl-CoA synthetase is preferably SEQ ID NO 6 or a variant thereof, or culturing the cell according to the first aspect or any embodiment thereof.

In a first embodiment of the second aspect, the method comprises, in addition to step b), the steps of
a) converting a fatty acid to an acyl CoA using an acyl-CoA synthetase, which may be preferably isolated and/or recombinant
and/or
c) hydrogenating.

In a second embodiment, which is also an embodiment of the first embodiment of the second aspect, at least one of the enzymes, preferably all of them, selected from the group consisting of amino acid-N-acyl-transferase and acyl-CoA synthetase may be provided in the form of a cell expressing said enzyme or enzymes.

In a third embodiment, which is also an embodiment of the first or second embodiments of the second aspect, the cell expressing said enzyme or enzymes may be the cell according to the first aspect or any embodiment thereof.

In a third aspect the present invention provides a reaction mixture comprising
an amino acid-N-acyl-transferase, which may be preferably isolated and/or recombinant,
an acyl-CoA synthetase, which may be preferably isolated and/or recombinant,
an amino acid and/or
either an acyl CoA or a fatty acid and an acyl-CoA-synthetase,
wherein the amino acid-N-acyl-transferase may preferably be a human amino acid-N-acyl-transferase, more preferably SEQ ID NO: 4, SEQ ID NO: 5 or a variant thereof, and wherein the acyl-CoA synthetase is preferably SEQ ID NO: 6 or a variant thereof.

In a first embodiment of the third aspect, at least one of the enzymes, preferably all of them, selected from the group consisting of an amino acid-N-acyl-transferase and an acyl-CoA synthetase may be provided in the form of a cell, preferably the cell according to the first aspect or any embodiment thereof.

In a second embodiment, which is also an embodiment of the first embodiment, the amino acid may be a proteinogenic amino acid, preferably selected from the group consisting of glycine, glutamine, glutamate, asparagine and alanine and may be more preferably glycine.

In a third embodiment, which is also an embodiment of the first to second embodiments, the fatty acid may be an unsaturated fatty acid and may be preferably selected from the group consisting of myristoleic acid, lauroleic acid, palmitoleic acid and cis-vaccenic acid.

In another embodiment of the second or third aspect, the fatty acid may be a saturated fatty acid and may be preferably selected from the group consisting of laurate, myristate and palmitate.

In another embodiment of the second or third aspect, the fatty acid may be provided in the form of an organic phase comprising a liquid organic solvent and the fatty acid, wherein the organic solvent may be preferably an ester of the fatty acid.

In a fourth aspect, the present invention provides a composition comprising
a first acyl amino acid comprising or consisting of a saturated acyl having 8 to 16 carbon atoms and glycine,
a second acyl amino acid comprising or consisting of an unsaturated acyl having 10 to 18 carbon atoms and glycine,
and optionally a third acyl amino acid comprising or consisting of a saturated or unsaturated acyl having 12 carbon atoms and an amino acid selected from the group consisting of glutamine, glutamic acid, alanine and asparagine.

In a first embodiment of the fourth aspect, the first acyl amino acid may comprise or consists of a saturated acyl having 12 carbon atoms, preferably lauryl, and glycine.

In a second embodiment of the fourth aspect, which is also an embodiment of the first embodiment of the fourth aspect, the second acyl amino acid may comprise or consists of an unsaturated acyl having 12 or 14 carbon atoms and glycine.

According to any aspect of the present invention, the acyl amino acids formed may be a mixture of amino acids. The mixture of amino acids may comprise at least two proteinogenic amino acids as defined below. In particular, the mixture of amino acids may have 3, 4, 5, 6, 7, 8, 9, 10, 11, 12, 13, 14, 15, 16, 17, 18, 19, 20, or 21 proteinogenic amino acids. In one example, the mixture of amino acids may be used to form cocoyl glycine and salts thereof.

The present invention is based on the surprising finding that the combination of amino acid-N-acyl transferase and an acyl-CoA synthetase, preferably expressed by a cell having reduced fatty acid degradation capacity, may be used to convert a variety of fatty acids, more preferably a mixture comprising unsaturated and saturated fatty acids, to acyl amino acids.

Moreover, the present invention is based on the surprising finding that there are amino acid-N-acyl transferases that may be used to convert short unsaturated fatty acids such as lauroleic acid to an acyl amino acid.

Moreover, the present invention is based on the surprising finding that employing an amino acid-N-acyl-transferase capable of converting a variety of fatty acids including short unsaturated fatty acids such as lauroleic acid to an acyl amino acid may increase the yields of acyl amino acids produced.

Moreover, the present invention is based on the surprising finding that the composition of acyl amino acids produced in a cell, more specifically the length of fatty acids incorporated into such acyl amino acids, may be controlled by introducing into the cell one or more specific acyl-CoA thioesterases or altering the expression of one or more acyl-CoA thioesterases endogenously expressed by the cell.

The present invention centres around the use of an amino acid-N-acyl transferase and an acyl-CoA synthetase for making acyl amino acids. In a preferred embodiment, the term “amino acid-N-acyl transferase”, as used herein, refers to an enzyme capable of catalysing the conversion of acyl-CoA, preferably the CoA ester of lauroleic acid, and an amino acid, preferably a proteinogenic amino acid, more preferably glycine, to an acyl amino acid. Suitable amino acid-N-acyl transferases have been described in the prior art, for example in Waluk, D. P., Schultz, N., and Hunt, M. C. (2010), Identification of glycine N-acyltransferase-like 2 (GLYATL2) as a transferase that produces N-acyl glycines in humans, FASEB J. 24, 2795-2803. In a preferred embodiment, the amino acid-N-acyl transferase comprises a nucleotide sequence of SEQ ID NO:4. In particular, the amino acid sequence of amino acid-N-acyl transferase may be selected from the group consisting of NP_001010904.1, NP_659453.3, XP_001147054.1, AAH16789.1, AAO73139.1, XP_003275392.1, XP_002755356.1, XP_003920208.1, XP_004051278.1, XP_006147456.1, XP_006214970.1, XP_003801413.1, XP_006189704.1, XP_003993512.1, XP_005862181.1, XP_007092708.1, XP_006772167.1, XP_006091892.1, XP_005660936.1, XP_005911029.1, NP_001178259.1, XP_004016547.1, XP_005954684.1, ELR45061.1, XP_005690354.1, XP_004409352.1, XP_007519553.1, XP_004777729.1, XP_005660935.1, XP_004824058.1, XP_006068141.1, XP_006900486.1, XP_007497585.1, XP_002821801.2, XP_007497583.1, XP_003774260.1, XP_001377648.2, XP_003909843.1, XP_003801448.1, XP_001091958.1, XP_002821798.1, XP_005577840.1, XP_001092197.1, NP_001207423.1, NP_001207425.1, XP_003954287.1, NP_001271595.1, XP_003909848.1, XP_004087850.1, XP_004051279.1, XP_003920209.1, XP_005577835.1, XP_003774402.1, XP_003909846.1, XP_004389401.1, XP_002821802.1, XP_003774401.1, XP_007497581.1, EHH21814.1, XP_003909845.1, XP_005577839.1, XP_003774403.1, XP_001092427.1, XP_003275395.2, NP_542392.2, XP_001147271.1, XP_005577837.1, XP_003826420.1, XP_004051281.1, XP_001147649.2, XP_003826678.1, XP_003909847.1, XP_004682812.1, XP_004682811.1, XP_003734315.1, XP_004715052.1, BAG62195.1, XP_003777804.1, XP_003909849.1, XP_001092316.2, XP_006167891.1, XP_540580.2, XP_001512426.1, EAW73833.1, XP_003464217.1, XP_007519551.1, XP_003774037.1, XP_005954680.1, XP_003801411.1, NP_803479.1, XP_004437460.1, XP_006875830.1, XP_004328969.1, XP_004264206.1, XP_004683490.1, XP_004777683.1, XP_005954681.1, XP_003480745.1, XP_004777682.1, XP_004878093.1, XP_007519550.1, XP_003421399.1, EHH53167.1, XP_006172214.1, XP_003993453.1, AAI12537.1, XP_006189705.1, Q2KIR7.2, XP_003421465.1, NP_001009648.1, XP_003464328.1, XP_001504745.1, ELV11036.1, XP_005690351.1, XP_005216632.1, EPY77465.1, XP_005690352.1, XP_004016544.1, XP_001498276.2, XP_004264205.1, XP_005690353.1, XP_005954683.1, XP_004667759.1, XP_004479306.1, XP_004645843.1, XP_004016543.1, XP_002928268.1, XP_006091904.1, XP_005331614.1, XP_007196549.1, XP_007092705.1, XP_004620532.1, XP_004869789.1, EHA98800.1, XP_004016545.1, XP_004479307.1, XP_004093105.1, NP_001095518.1, XP_005408101.1, XP_004409350.1, XP_001498290.1, XP_006056693.1, XP_005216639.1, XP_007455745.1, XP_005352049.1, XP_004328970.1, XP_002709220.1, XP_004878092.1, XP_007196553.1, XP_006996816.1, XP_005331615.1, XP_006772157.1, XP_007196552.1, XP_004016546.1, XP_007628721.1, NP_803452.1, XP_004479304.1, DAA21601.1, XP_003920207.1, XP_006091906.1, XP_003464227.1, XP_006091903.1, XP_006189706.1, XP_007455744.1, XP_004585544.1, XP_003801410.1, XP_007124812.1, XP_006900488.1, XP_004777680.1, XP_005907436.1, XP_004389356.1, XP_007124811.1, XP_005660937.1, XP_007628724.1, XP_003513512.1, XP_004437813.1, XP_007628723.1, ERE78858.1, EPQ15380.1, XP_005862178.1, XP_005878672.1, XP_540581.1, XP_002928267.1, XP_004645845.1, EPQ05184.1, XP_003513511.1, XP_006214972.1, XP_007196545.1, XP_007196547.1, XP_006772160.1, XP_003801409.1, NP_001119750.1, XP_003801412.1, XP_006772159.1, EAW73832.1, XP_006091897.1, XP_006772163.1, XP_006091898.1, XP_005408105.1, XP_006900487.1, XP_003993454.1, XP_003122754.3, XP_007455746.1, XP_005331618.1, XP_004585337.1, XP_005063305.1, XP_006091895.1, XP_006772156.1, XP_004051276.1, XP_004683488.1, NP_666047.1, NP_001013784.2, XP_006996815.1, XP_006996821.1, XP_006091893.1, XP_006173036.1, XP_006214971.1, EPY89845.1, XP_003826423.1, NP_964011.2, XP_007092707.1, XP_005063858.1, BAL43174.1, XP_001161154.2, XP_007124813.1, NP_083826.1 XP_003464239.1, XP_003275394.1, ELK23978.1, XP_004878097.1, XP_004878098.1, XP_004437459.1, XP_004264204.1, XP_004409351.1, XP_005352047.1, Q5RFP0.1, XP_005408107.1, XP_007659164.1, XP_003909852.1, XP_002755355.1, NP_001126806.1, AAP92593.1, NP_001244199.1, BAA34427.1, XP_005063859.1, NP599157.2, XP_004667761.1, XP_006900489.1, XP_006215013.1, XP_005408100.1, XP_007628718.1, XP_003514769.1, XP_006160935.1, XP_004683489.1, XP_003464329.1, XP_004921258.1, XP_003801447.1, XP_006167892.1, XP_004921305.1, AAH89619.1, XP_004706162.1, XP_003583243.1, EFB16804.1, XP_006728603.1, EPQ05185.1, XP_002709040.1, XP_006875861.1, XP_005408103.1, XP_004391425.1, EDL41477.1, XP_006772158.1, EGW06527.1, AAH15294.1, XP_006772162.1, XP_005660939.1, XP_005352050.1, XP_006091901.1, XP_005878675.1, XP_004051323.1, EHA98803.1, XP_003779925.1, EDM12924.1, XP_003421400.1, XP_006160939.1, XP_006160938.1, XP_006160937.1, XP_006160936.1, XP_005702185.1, XP_005313023.1, XP_003769190.1, XP_002714424.1, XP_004715051.1, XP_007661593.1, XP_004590594.1, ELK23975.1, XP_004674085.1, XP_004780477.1, XP_006231186.1, XP_003803573.1, XP_004803176.1, EFB16803.1, XP_006056694.1, XP_005441626.1, XP_005318647.1XP_004605904.1, XP_005862182.1, XP_003430682.1, XP_004780478.1, XP_005239278.1, XP_003897760.1, XP_007484121.1, XP_004892683.1, XP_004414286.1, XP_006927013.1, XP_003923145.1, XP_852587.2, AAP97178.1, EHH53105.1, XP_005408113.1, XP_002915474.1, XP_005377590.1, XP_527404.2, XP_005552830.1, XP_004044211.1, NP_001180996.1, XP_003513513.2, XP_001498599.2, XP_002746654.1, XP_005072349.1, XP_006149181.1, EAX04334.1, XP_003833230.1, XP_005216635.1, XP_003404197.1, XP_007523363.1, XP_007433902.1, XP_003254235.1, XP_004471242.1, XP_005216634.1, XP_006860675.1, XP_004771956.1, XP_006038833.1, NP_001138534.1, XP_007068532.1, XP_003510714.1, ERE87950.1, XP_003986313.1, XP_006728644.1, XP_004878099.1, XP_003468014.1, XP_007095614.1, XP_004648849.1, XP_004869795.1, XP_004018927.1, XP_005696454.1, XP_006201985.1, XP_005960697.1, XP_004813725.1, XP_005496926.1, ELR45088.1, XP_004696625.1, XP_005860982.1, XP_005911003.1, XP_006260162.1, EPQ04414.1, XP_006099775.1, NP_001138532.1, XP_006190795.1, XP_004649775.1, XP_004424497.1, XP_004390885.1, XP_005911004.1, XP_003777803.1, XP_004312259.1, XP_005529140.1, XP_005314582.1, XP_006926523.1, XP_006926522.1, XP_004683491.1, XP_003826680.1, XP_003215018.1, XP_003215087.1, EGW12611.1, XP_006113023.1, XP_006882182.1, XP_007425200.1, XP_006041342.1, NP_001138533.1, EMP27694.1, XP_007497753.1, XP_006034252.1, or a variant thereof. Throughout this application, any data base code, unless specified to the contrary, refers to a sequence available from the NCBI data bases, more specifically the version online on 5 Aug. 2013, and comprises, if such sequence is a nucleotide sequence, the polypeptide sequence obtained by translating the former.

In a preferred embodiment, the term “acyl amino acid”, as used herein, refers to the product of the reaction catalysed by an amino acid-N-acyl transferase, more preferably a compound represented by the formula acyl-CO—NH—CHR—COOH, wherein R is the side chain of a proteinogenic amino acid, and wherein the term “acyl” refers to the acyl residue of a fatty acid. In a preferred embodiment of the present invention, the term “fatty acid”, as used herein, means a carboxylic acid, preferably alkanoic acid, with at least 6, preferably 8, more preferably 10, most preferably 12 carbon atoms. In a preferred embodiment it is a linear fatty acid, in another embodiment it is branched. In a preferred embodiment it is a saturated fatty acid. In an especially preferred embodiment it is unsaturated. In another preferred embodiment it is a linear fatty acid with at least 12 carbon atoms comprising a double bond, preferably at position 9. In another preferred embodiment it is a simple unsaturated fatty acid having one double bond, which double bond is located at position 9 or 11. In the most preferred embodiment it is lauroleic acid (9-dodecenoic acid). In an especially preferred embodiment it is a fatty acid with 6, 7, 8, 9, 10, 11, 12, 13, 14, 15, 16, 17, 18, 19, 20, 21, 22, 23, 24, 25, 26, 27, 28, 29, 30 carbon atoms, preferably 12 carbon atoms.

Throughout this application, a formula referring to a chemical group that represents the dissociated or undissociated state of a compound capable of dissociating in an aqueous solution, comprises both the dissociated and the undissociated state and the various salt forms of the group. For example, the residue —COOH comprises both the protonated (—COOH) as well as the unprotonated (—COO⁻) carboxylic acid.

The acyl-CoA substrate consumed in the inventive reaction may be purified from a cell, chemically synthesised or produced using an acyl-CoA synthetase, the latter being the preferred option. In a preferred embodiment, the term “acyl-CoA synthetase”, as used herein, refers to an enzyme capable of catalysing the ATP-dependent conversion of a fatty acid and CoA to acyl CoA.

According to any aspect of the present invention, the term ‘acyl-CoA synthetase’ may refer to an acyl-CoA/ACP synthetase that may be capable of producing acyl glycinates and/or catalysing the following reaction:

fatty acid+CoA/ACP+ATP→acyl-CoA/ACP+ADP+Pi

Examples of acyl-CoA/ACP synthetases may include EC 6.2.1.3, EC 6.2.1.10, EC 6.2.1.15, EC 6.2.1.20 and the like. The state of the art describes various methods to detect acyl-CoA synthetase activity. For example, the activity of an acyl-CoA synthetase may be assayed by incubating the sample of interest in 100 mM Tris-HCl at pH 8 in the presence of 250 μM lauroyl-CoA, 500 μM glycine and DTNB (5,5′-dithiobis-2-nitrobenzoic acid, also referred to as Ellman's reagent) and spectrophotometrically monitoring the absorbance at 410 nm following release of free thiol groups in the form of CoASH as the reaction progresses and reaction with Ellman's reagent.

Various acyl-CoA synthetases have been described in the state of the art, for example YP_001724804.1, WP_001563489.1 and NP_707317.1. In a preferred embodiment, the acyl-CoA synthetase comprises SEQ ID NO 6 or YP_001724804.1 or a variant thereof. The activity of an acyl-CoA synthetase may be assayed as described in the state of the art, for example Kang, Y., Zarzycki-Siek, J., Walton, C. B., Norris, M. H., and Hoang, T. T. (2010), Multiple FadD Acyl-CoA Synthetases Contribute to Differential Fatty Acid Degradation and Virulence in Pseudomonas aeruginosa, PLOS ONE 5 (10), e13557. Briefly, the amount of free thiol in the form of unreacted CoASH is determined by adding Ellmann's reagent and spectrophotometrically monitoring the absorbance at 410 nm, preferably in a reaction buffer comprising 150 mM Tris-HCl (pH 7.2), 10 mM MgCl₂, 2 mM EDTA, 0.1% Triton X-100, 5 mM ATP, 0.5 mM coenzyme A (CoASH) and a fatty acid (30 to 300 mM).

In one example, the cell according to any aspect of the present invention may be genetically modified to overexpress at least the enzymes amino acid-N-acyl transferase and acyl-CoA synthetase. In particular, the cell may over express enzymes glycine N-acyl transferase and acyl-CoA/ACP synthetase.

The teachings of the present invention may not only be carried out using biological macromolecules having the exact amino acid or nucleic acid sequences referred to in this application explicitly, for example by name or accession number, or implicitly, but also using variants of such sequences. In a preferred embodiment, the term “variant”, as used herein, comprises amino acid or nucleic acid sequences, respectively, that are at least 70, 75, 80, 85, 90, 92, 94, 95, 96, 97, 98 or 99% identical to the reference amino acid or nucleic acid sequence, wherein preferably amino acids other than those essential for the function, for example the catalytic activity of a protein, or the fold or structure of a molecule are deleted, substituted or replaced by insertions or essential amino acids are replaced in a conservative manner to the effect that the biological activity of the reference sequence or a molecule derived therefrom is preserved. The state of the art comprises algorithms that may be used to align two given nucleic acid or amino acid sequences and to calculate the degree of identity, see Arthur Lesk (2008), Introduction to bioinformatics, 3^{rd} edition, Thompson et al., Nucleic Acids Research 22, 4637-4680, 1994, and Katoh et al., Genome Information, 16(1), 22-33, 2005. The term “variant” is used synonymously and interchangeably with the term “homologue”. Such variants may be prepared by introducing deletions, insertions or substitutions in amino acid or nucleic acid sequences as well as fusions comprising such macromolecules or variants thereof. In a preferred embodiment, the term “variant”, with regard to amino acid sequence, comprises, preferably in addition to the above sequence identity, amino acid sequences that comprise one or more conservative amino acid changes with respect to the respective reference or wild type sequence or comprises nucleic acid sequences encoding amino acid sequences that comprise one or more conservative amino acid changes. In a preferred embodiment, the term “variant” of an amino acid sequence or nucleic acid sequence comprises, preferably in addition to the above degree of sequence identity, any active portion and/or fragment of the amino acid sequence or nucleic acid sequence, respectively, or any nucleic acid sequence encoding an active portion and/or fragment of an amino acid sequence. In a preferred embodiment, the term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity. For example an active portion and/or fragment of a protease is capable of hydrolysing peptide bonds in polypeptides. In a preferred embodiment, the term “retains at least some of its essential biological activity”, as used herein, means that the amino acid sequence in question has a biological activity exceeding and distinct from the background activity and the kinetic parameters characterising said activity, more specifically k_{cat} and K_{M}, are preferably within 3, more preferably 2, most preferably one order of magnitude of the values displayed by the reference molecule with respect to a specific substrate. In a preferred embodiment, the term “variant” of a nucleic acid comprises nucleic acids the complementary strand of which hybridises, preferably under stringent conditions, to the reference or wild type nucleic acid.

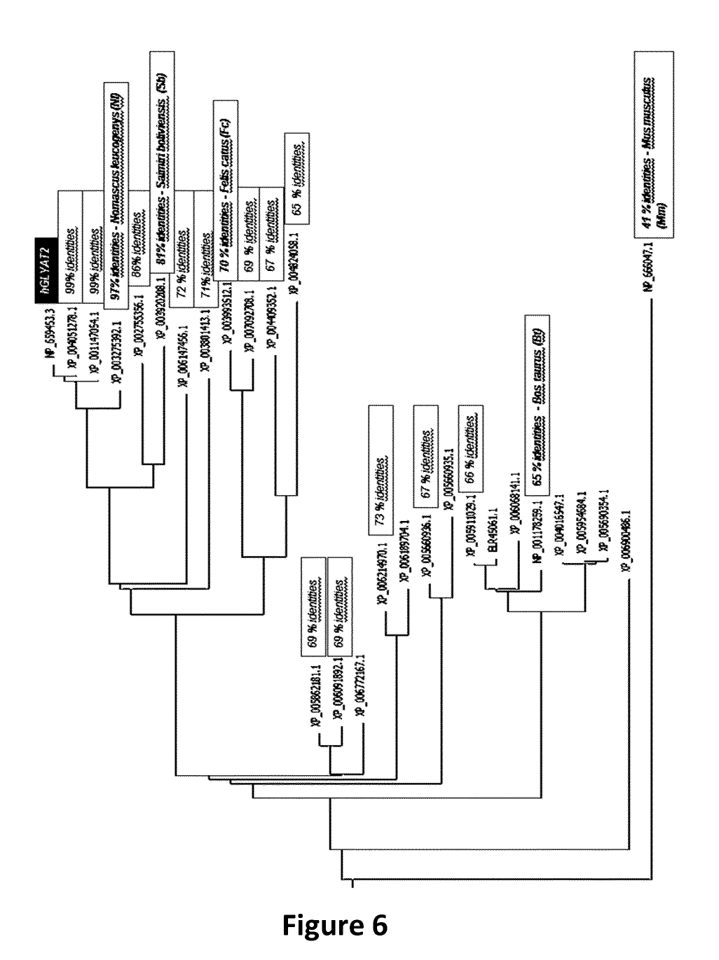

Examples of variants of amino acid-N-acyl-transferases may at least be provided in FIG. 6. A skilled person would be able to easily determine the amino acid-N-acyl-transferases that will be capable of making proteinogenic amino acids and/or fatty acids. In particular, the variants may include but are not limited to an amino acid-N-acyl-transferase selected from the group of organisms consisting of Nomascus leucogenys (NI, XP_003275392.1, SEQ ID No: 53), Saimiri boliviensis (Sb, XP_003920208.1, SEQ ID No: 54), Felis catus (Fc, XP_003993512.1, SEQ ID No 55), Bos taurus (Bt, NP_{—} 001178259.1, SEQ ID No: 56), Mus musculus (Mm, NP_666047.1, SEQ ID No: 57).

Stringency of hybridisation reactions is readily determinable by one of ordinary skilled in the art, and generally is an empirical calculation dependent on probe length, washing temperature and salt concentration. In general, longer probes require higher temperatures for proper annealing, while shorter probes need lower temperatures. Hybridisation generally depends on the ability of denatured DNA to reanneal to complementary strands when present in an environment below their melting temperature. The higher the degree of desired homology between the probe and hybridisable sequence, the higher the relative temperature which may be used. As a result it follows that higher relative temperatures would tend to make the reaction conditions more stringent, while lower temperature less so. For additional details and explanation of stringency of hybridisation reactions, see F. M. Ausubel (1995), Current Protocols in Molecular Biology. John Wiley & Sons, Inc. Moreover, the person skilled take in the art may follow the instructions given in the manual “The DIG System Users Guide for Filter Hybridization”, Boehringer Mannheim GmbH, Mannheim, Germany, 1993 and in Liebl et al. (International Journal of Systematic Bacteriology 41: 255-260 (1991) on how to identify DNA sequences by means of hybridisation. In a preferred embodiment, stringent conditions are applied for any hybridisation, i.e. hybridisation occurs only if the probe is 70% or more identical to the target sequence. Probes having a lower degree of identity with respect to the target sequence may hybridise, but such hybrids are unstable and will be removed in a washing step under stringent conditions, for example lowering the concentration of salt to 2×SSC or, optionally and subsequently, to 0.5×SSC, while the temperature is, in order of increasing preference, approximately 50° C.-68° C., approximately 52° C.-68° C., approximately 54° C.-68° C., approximately 56° C.-68° C., approximately 58° C.-68° C., approximately 60° C.-68° C., approximately 62° C.-68° C., approximately 64° C.-68° C., approximately 66° C.-68° C. In a particularly preferred embodiment, the temperature is approximately 64° C.-68° C. or approximately 66° C.-68° C. It is possible to adjust the concentration of salt to 0.2×SSC or even 0.1×SSC. Polynucleotide fragments having a degree of identity with respect to the reference or wild type sequence of at least 70, 80, 90, 91, 92, 93, 94, 95, 96, 97, 98, 99% may be isolated. In a preferred embodiment, the term “homologue” of a nucleic acid sequence, as used herein, refers to any nucleic acid sequence that encodes the same amino acid sequence as the reference nucleic acid sequence, in line with the degeneracy of the genetic code.

If one or more of the enzymes used to practise the present invention is provided in the form of a cell, it is preferred that the cell has a reduced fatty acid degradation capacity. In a preferred embodiment, the term “having a reduced fatty acid degradation capacity”, as used herein, means that the respective cell degrades fatty acids, preferably those taken up from the environment, at a lower rate than a comparable cell or wild type cell having normal fatty acid degradation capacity would under identical conditions. In a preferred embodiment, the fatty acid degradation of such a cell is lower on account of deletion, inhibition or inactivation of at least one gene encoding an enzyme involved in the β-oxidation pathway. In a preferred embodiment of the present invention, at least one enzyme involved in the β-oxidation pathway has lost, in order of increasing preference, 5, 10, 20, 40, 50, 75, 90 or 99% activity relative to the activity of the same enzyme under comparable conditions in the respective wild type microorganism. The person skilled in the art is familiar with various techniques that may be used to delete a gene encoding an enzyme or reduce the activity of such an enzyme in a cell, for example by exposition of cells to radioactivity followed by accumulation or screening of the resulting mutants, site-directed introduction of point mutations or knock out of a chromosomally integrated gene encoding for an active enzyme, as described in Sambrook/Fritsch/Maniatis (1989). In addition, the transcriptional repressor FadR may be over expressed to the effect that expression of enzymes involved in the β-oxidation pathway is repressed (Fujita, Y., Matsuoka, H., and Hirooka, K. (2007) Mol. Microbiology 66(4), 829-839). In a preferred embodiment, the term “deletion of a gene”, as used herein, means that the nucleic acid sequence encoding said gene is modified such that the expression of active polypeptide encoded by said gene is reduced. For example, the gene may be deleted by removing in-frame a part of the sequence comprising the sequence encoding for the catalytic active centre of the polypeptide. Alternatively, the ribosome binding site may be altered such that the ribosomes no longer translate the corresponding RNA. Moreover, the person skilled in the art is able to routinely measure the activity of enzymes expressed by living cells using standard essays as described in enzymology text books, for example Cornish-Bowden (1995), Fundamentals of Enzyme Kinetics, Portland Press Limited, 1995.

Degradation of fatty acids is accomplished by a sequence of enzymatically catalysed reactions. First of all, fatty acids are taken up and translocated across the cell membrane via a transport/acyl-activation mechanism involving at least one outer membrane protein and one inner membrane-associated protein which has fatty acid-CoA ligase activity, referred to in the case of E. coli as FadL and FadD/FadK, respectively. Inside the cell, the fatty acid to be degraded is subjected to enzymes catalysing other reactions of the β-oxidation pathway. The first intracellular step involves the conversion of acyl-CoA to enoyl-CoA through acyl-CoA dehydrogenase, the latter referred to as FadE in the case of E. coli. The activity of an acyl-CoA dehydrogenase may be assayed as described in the state of art, for example by monitoring the concentration of NADH spectrophotometrically at 340 nm in 100 mM MOPS, pH 7.4, 0.2 mM Enoyl-CoA, 0.4 mM NAD⁺. The resulting enoyl-CoA is converted to 3-ketoacyl-CoA via 3-hydroxylacyl-CoA through hydration and oxidation, catalysed by enoyl-CoA hydratase/3-hydroxyacyl-CoA dehydrogenase, referred to as FadB and FadJ in E. coli. Enoyl-CoA hydratase/3-hydroxyacyl-CoA dehydrogenase activity, more specifically formation of the product NADH may be assayed spectrophotometrically as described in the state of the art, for example as outlined for FadE. Finally, 3-ketoacyl-CoA thiolase, FadA and Fadl in E. coli, catalyses the cleavage of 3-ketoacyl-CoA, to give acetyl-CoA and the input acyl-CoA shortened by two carbon atoms. The activity of ketoacyl-CoA thiolase may be assayed as described in the state of the art, for example in Antonenkov, V., D. Van Veldhoven, P., P., Waelkens, E., and Mannaerts, G.P. (1997) Substrate specificities of 3-oxoacyl-CoA thiolase and sterol carrier protein 2/3-oxoacyl-coa thiolase purified from normal rat liver peroxisomes. Sterol carrier protein 2/3-oxoacyl-CoA thiolase is involved in the metabolism of 2-methyl-branched fatty acids and bile acid intermediates. J. Biol. Chem. 1997, 272:26023-26031. In a preferred embodiment, the term “a cell having a reduced fatty acid degradation capacity”, as used herein, refers to a cell having a reduced capability of taking up and/or degrading fatty acids, preferably those having at least eight carbon chains. The fatty acid degradation capacity of a cell may be reduced in various ways. In a preferred embodiment, the cell has, compared to its wild type, a reduced activity of an enzyme involved in the β-oxidation pathway. In a preferred embodiment, the term “enzyme involved in the β-oxidation pathway”, as used herein, refers to an enzyme that interacts directly with a fatty acid or a derivative thereof formed as part of the degradation of said fatty acid via the β-oxidation pathway the sequence of reactions effecting the conversion of a fatty acid to acetyl-CoA and the CoA ester of the shortened fatty acid, preferably by recognizing the fatty acid or derivative thereof as a substrate, and converts it to a metabolite formed as a part of the β-oxidation pathway. For example, the acyl-CoA dehydrogenase is an enzyme involved in the β-oxidation pathway as it interacts with fatty acid-CoA and converts fatty acid-CoA ester to enoyl-CoA, which is a metabolite formed as part of the β-oxidation. In a preferred embodiment, the term “enzyme involved in the β-oxidation pathway”, as used herein, comprises any polypeptide from the group comprising acyl-CoA dehydrogenase, enoyl-CoA hydratase, 3-hydroxyacyl-CoA dehydrogenase and 3-keto-acyl-CoA thiolase. Subsequently, the acyl-CoA synthetase may catalyse the conversion of a fatty acid to the CoA ester of a fatty acid, i.e. a molecule, wherein the functional group —OH of the carboxy group is replaced with —S-CoA, preferably for introducing said fatty acid into the β-oxidation pathway. For example, the polypeptides FadD and FadK in E. coli (accession number: BAA15609.1) are acyl-CoA dehydrogenases. In a preferred embodiment, the term “acyl-CoA dehydrogenase”, as used herein, is a polypeptide capable of catalysing the conversion of an acyl-CoA to enoyl-CoA, preferably as part of the β-oxidation pathway. For example, the polypeptide FadE in E. coli (accession number: BAA77891.2) is an acyl-CoA dehydrogenase. In a preferred embodiment, the term “2,4-dienoyl-CoA reductase”, as used herein, is a polypeptide capable of catalysing the conversion of the 2,4-dienoyl CoA from an unsaturated fatty acid into enoyl-CoA, preferably as part of the β-oxidation pathway. For example, the polypeptide FadH in E. coli is a 2,4-dienoyl-CoA reductase. In a preferred embodiment, the term “enoyl-CoA hydratase”, as used herein, also referred to as 3-hydroxyacyl-CoA dehydrogenase, refers to a polypeptide capable of catalysing the conversion of enoyl-CoA to 3-ketoacyl-CoA through hydration and oxidation, preferably as part of the β-oxidation pathway. For example, the polypeptides FadB and FadJ in E. coli (accession number: BAE77457.1) are enoyl-CoA hydratases. In a preferred embodiment, the term “ketoacyl-CoA thiolase”, as used herein, refers to a polypeptide capable of catalysing the conversion of cleaving 3-ketoacyl-CoA, resulting in an acyl-CoA shortened by two carbon atoms and acetyl-CoA, preferably as the final step of the β-oxidation pathway. For example, the polypeptides FadA and Fadl in E. coli (access code: AP009048.1) are ketoacyl-CoA thiolases.

In one example, the cells according to any aspect of the present invention may be genetically modified to result in an increased activity of at least one amino acid N-acyl transferase in combination with increased activity of at least one acyl-CoA synthetase in combination with an increased activity of at least one transporter protein of the FadL and/or the AlkL. In particular, the cell according to any aspect of the present invention may be genetically modified to overexpress glycine N-acyl transferase, acyl-CoA/ACP synthetase and a transporter protein of the FadL and/or the AlkL compared to the wild type cell. These cells may be capable of producing acyl glycinates. In another example, the cell according to any aspect of the present invention may be genetically modified compared to the wild type cell to:
increase the expression of amino acid N-acyl transferase, acyl-CoA synthetase; and
reduce activity of at least one enzyme selected from the group consisting of acyl-CoA dehydrogenase Fad E, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, electron-transfer flavoprotein.

In yet another example, the cells according to any aspect of the present invention may be genetically modified compared to the wild type cell to result in:
increase in the expression of amino acid N-acyl transferase, acyl-CoA synthetase and at least one transporter protein of the FadL and/or the AlkL; and
reduce activity of at least one enzyme selected from the group consisting of acyl-CoA dehydrogenase Fad E, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, electron-transfer flavoprotein.

In particular, the acyl-CoA dehydrogenase FadE (EC 1.3.8.7, EC 1.3.8.8 or EC 1.3.8.9) may catalyse the reaction acyl-CoA+electron-transfer flavoprotein=trans-2,3-dehydroacyl-CoA+reduced electron-transfer flavoprotein; the multifunctional 3-hydroxybutyryl-CoA epimerase (EC 5.1.2.3), Δ3-cis-Δ2-trans-enoyl-CoA isomerase (EC 5.3.3.8), enoyl-CoA hydratase (EC 4.2.1.17) and 3-hydroxyacyl-CoA dehydrogenase (EC 1.1.1.35) FadB, may catalyse the reactions (S)-3-hydroxybutanoyl-CoA→(R)-3-hydroxybutanoyl-CoA, (3Z)-3-enoyl-CoA→(2E)-2-enoyl-CoA, (3S)-3-hydroxyacyl-CoA→trans-2-enoyl-CoA+H2O, (S)-3-hydroxyacyl-CoA+NAD+=3-oxoacyl-CoA+NADH+H+; 3-ketoacyl-CoA thiolase (EC 2.3.1.16), may catalyse the reaction acyl-CoA+acetyl-CoA→CoA+3-oxoacyl-CoA; and electron-transfer flavoprotein (EC 1.5.5.1), may catalyse the following reaction: reduced electron-transferring flavoprotein+ubiquinone 4 electron-transferring flavoprotein+ubiquinol.

More in particular, the cell according to any aspect of the present invention may be genetically modified compared to the wild-type of the cell to increase the expression of glycine N-acyl transferase, acyl-CoA/ACP synthetase, a transporter protein of the FadL and the AlkL; and reduce activity of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase Fad B, 3-ketoacyl-CoA thiolase, electron-transfer flavoprotein.

In one example, the cell according to any aspect of the present invention may be genetically modified compared to the wild-type of the cell to:
increase the expression of amino acid N-acyl transferase, acyl-CoA synthetase; and
reduce activity of at least one enzyme selected from the group consisting of glycine cleavage system H protein, glycine cleavage system P protein, glycine cleavage system L protein, glycine cleavage system T protein, threonine aldolase, and serine hydroxylmethyltransferase.

In another example, the cell according to any aspect of the present invention may be genetically modified compared to the wild-type of the cell to:
increase the expression of amino acid N-acyl transferase, acyl-CoA synthetase and at least one transporter protein of the FadL and/or the AlkL; and
reduce activity of at least one enzyme selected from the group consisting of acyl-CoA dehydrogenase Fad E, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, and electron-transfer flavoprotein.

In yet another example, the cell according to any aspect of the present invention may be genetically modified compared to the wild-type of the cell to:
increase the expression of amino acid N-acyl transferase, acyl-CoA synthetase and at least one transporter protein of the FadL and/or the AlkL;
reduce activity of at least one enzyme selected from the group consisting of acyl-CoA dehydrogenase Fad E, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, and electron-transfer flavoprotein; and
reduce activity of at least one enzyme selected from the group consisting of glycine cleavage system H protein, glycine cleavage system P protein, glycine cleavage system L protein, glycine cleavage system T protein, threonine aldolase, and serine hydroxylmethyltransferase.

In particular, the cell according to any aspect of the present invention may be genetically modified compared to the wild-type of the cell to increase the expression of glycine N-acyl transferase, acyl-CoA/ACP synthetase, a transporter protein of the FadL and the AlkL; reduce activity of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, electron-transfer flavoprotein; and reduce activity of glycine cleavage system H protein, glycine cleavage system P protein, glycine cleavage system L protein, glycine cleavage system T protein, threonine aldolase, and serine hydroxylmethyltransferase.

More in particular, the glycine cleavage system H protein, carries lipoic acid and interacts with the glycine cleavage system proteins P, L and T; the glycine cleavage system P protein (EC 1.4.4.2), catalyses the reaction glycine+[glycine-cleavage complex H protein]-N(6)-lipoyl-L-lysine→[glycine-cleavage complex H protein]-S-aminomethyl-N(6)-dihydrolipoyl-L-lysine+CO2; glycine cleavage system L protein (EC 1.8.1.4), catalyses the reaction protein N6-(dihydrolipoyl)lysine+NAD+→protein N6-(lipoyl)lysine+NADH+H+; glycine cleavage system T protein (EC 2.1.2.10), catalyses the reaction [protein]-S8-aminomethyldihydrolipoyllysine+tetrahydrofolate→[protein]-dihydrolipoyllysine+5,10-methylenetetrahydrofolate+NH3; threonine aldolase (EC 4.2.1.48), catalyses the reaction L-threonine→glycine+acetaldehyde; serine hydroxylmethyltransferase (EC 2.1.2.1), catalyses the reaction 5,10-methylenetetrahydrofolate+glycine+H₂O+tetrahydrofolate+L-serine.

In one example, the cell according to any aspect of the present invention may be genetically modified compared to the wild-type of the cell to increase the expression of amino acid N-acyl transferase, acyl-CoA synthetase, and a genetic modification in the cell capable of producing at least one fatty acid from at least one carbohydrate. A list of non-limiting genetic modification to enzymes or enzymatic activities is provided below in Table 1. The cells according to any aspect of the present invention may comprise a combination of genetic modification that produce fatty acids and convert the fatty acids to N-acyl amino acids. In particular, the cell according to any aspect of the present invention may be genetically modified to increase the expression of amino acid N-acyl transferase, acyl-CoA synthetase and comprise any of the genetic modifications listed in Table 1. More in particular, the cell may be genetically modified to increase the expression of N-acyl transferase, acyl-CoA synthetase, a transporter protein of the FadL and the AlkL and comprise any of the genetic modifications listed in Table 1. Even more in particular, the cell may be genetically modified to increase the expression of N-acyl transferase, acyl-CoA synthetase, a transporter protein of the FadL and the AlkL, reduce activity of acyl-CoA dehydrogenase FadE, multifunctional 3-hydroxybutyryl-CoA epimerase, Δ3-cis-Δ2-trans-enoyl-CoA isomerase, enoyl-CoA hydratase, 3-hydroxyacyl-CoA dehydrogenase FadB, 3-ketoacyl-CoA thiolase, electron-transfer flavoprotein; reduce activity of glycine cleavage system H protein, glycine cleavage system P protein, glycine cleavage system L protein, glycine cleavage system T protein, threonine aldolase, serine hydroxylmethyltransferase, and comprise any of the genetic modifications listed in Table 1.

In one example, the cell according to any aspect of the present invention may be genetically modified to increase the expression of amino acid N-acyl transferase, acyl-CoA/ACP synthetase and decrease the expression of at least one enzyme selected from the group consisting of β-ketoacyl-ACP synthase I, 3-oxoacyl-ACP-synthase I, Malonyl-CoA-ACP transacylase, enoyl ACP reductase, and β-ketoacyl-ACP synthase III. In particular, the genetic modification in the cell according to any aspect of the present invention may comprise an increase in the expression of amino acid N-acyl transferase, acyl-CoA/ACP synthetase and a decrease in the expression of β-ketoacyl-ACP synthase I, 3-oxoacyl-ACP-synthase I, Malonyl-CoA-ACP transacylase, enoyl ACP reductase, and β-ketoacyl-ACP synthase III.

The inventive teachings may be carried out using a wide range of cells. In a preferred embodiment, the term “cell”, as used herein, refers to any permanently unicellular organism comprising bacteria archaea, fungi, algae and the like. In a preferred embodiment, the cell is a bacterial cell, more preferably one from the group comprising Pseudomonas, Corynebacterium, Bacillus and Escherichia, most preferably Escherichia coli. In another preferred embodiment, the cell is a lower eukaryote, more preferably a fungus from the group comprising Saccharomyces, Candida, Pichia, Schizosaccharomyces and Yarrowia, and is most preferably Saccharomyces cerevisiae. The microorganism may be an isolated cell, in other words a pure culture of a single strain, or may comprise a mixture of at least two strains. Biotechnologically relevant cells are commercially available, for example from the American Type Culture Collection (ATCC) or the German Collection of Microorganisms and Cell Cultures (DSMZ). Particles for keeping and modifying cells are available from the prior art, for example Sambrook/Fritsch/Maniatis (1989): Molecular cloning—A Laboratory Manual, Cold Spring Harbour Press, 2^{nd} edition, Fuchs/Schlegel (2007), Allgemeine Mikrobiologie, 2008, Georg Thieme Verlag.

Although the inventive teachings may be practiced using wild type cells, it is preferred that at least one of the enzymes involved, in particular at least one or all from the group comprising amino acid amino acid N-acyl-transferase, acyl-CoA synthetase and acyl-CoA thioesterase, is recombinant. In a preferred embodiment, the term “recombinant” as used herein, refers to a molecule or is encoded by such a molecule, preferably a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

Whether or not a nucleic acid molecule, polypeptide, more specifically an enzyme required to practice the invention, is recombinant or not has not necessarily implications for the level of its expression. However, it is preferred that one or more recombinant nucleic acid molecules, polypeptides or enzymes required to practice the invention are overexpressed. In a preferred embodiment, the term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell. The person skilled in the art is familiar with numerous ways to bring about overexpression. For example, the nucleic acid molecule to be overexpressed or encoding the polypeptide or enzyme to be overexpressed may be placed under the control of a strong inducible promoter such as the lac promoter. The state of the art describes standard plasmids that may be used for this purpose, for example the pET system of vectors exemplified by pET-3a (commercially available from Novagen). Whether or not a nucleic acid or polypeptide is overexpressed may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions. Thus, in various examples of the present invention, to function more efficiently, a microorganism may comprise one or more gene deletions. Gene deletions may be accomplished by mutational gene deletion approaches, and/or starting with a mutant strain having reduced or no expression of one or more of these enzymes, and/or other methods known to those skilled in the art.

Besides, any of the enzymes required to practice the inventive teachings, in particular at least one or all from the group comprising N-acyl-transferase, acyl-CoA synthetase and acyl-CoA thioesterase, may be an isolated enzyme. In any event, any enzyme required to practice the present invention is preferably used in an active state and in the presence of all cofactors, substrates, auxiliary and/or activating polypeptides or factors essential for its activity. In a preferred embodiment, the term “isolated”, as used herein, means that the enzyme of interest is enriched compared to the cell in which it occurs naturally. Whether or not an enzyme is enriched may be determined by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye.

If a cell expressing an amino acid-N-acyl-transferase and an acyl-CoA synthetase and having a reduced fatty acid degradation capacity is used, it is preferred that the cell be capable of making proteinogenic amino acids and/or fatty acids. In a preferred embodiment, the term “proteinogenic amino acid”, as used herein, refers to an amino acid selected from the group comprising alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. Proteinogenic amino acids and fatty acids are synthesized as part of the primary metabolism of many wild type cells in reactions and using enzymes that have been described in detail in biochemistry textbooks, for example Jeremy M Berg, John L Tymoczko, and Lubert Stryer, Biochemistry, 5^{th} edition, W.H. Freeman, 2002.

In a preferred embodiment, the cell used to practice the inventive teachings expresses an acyl-CoA thioesterase. In a more preferred embodiment, the term “acyl-CoA thioesterase”, as used herein, refers to an enzyme capable of hydrolysing acyl-CoA. In a preferred embodiment the acyl-CoA thioesterase comprises a sequence from the group comprising SEQ ID NO 1, AEM72521.1 and AAC49180.1 or a variant thereof, more preferably SEQ ID NO 1 or a variant thereof. The activity of acyl-CoA thioesterase may be assayed using various assays described in the state of the art. Briefly, the reaction of Ellman's reagent, which reacts with free thiol groups associated with CoASH formed upon hydrolysis of acyl-CoA may be detected by spectophotometrically monitoring absorbance at 412 nm.

In a preferred embodiment, the term “contacting”, as used herein, means bringing about direct contact between the amino acid, the acyl CoA and the amino acid-N-acyl transferase or the inventive cell and/or any other reagents required to carry out the inventive teachings, preferably in an aqueous solution. For example, the cell, the amino acid and the acyl CoA may not be in different compartments separated by a barrier such as an inorganic membrane. If the amino acid or fatty acid is soluble and may be taken up by the cell or can diffuse across biological membranes, it may simply be added to the inventive cell in an aqueous solution. In case it is insufficiently soluble, it may be solved in a suitable organic solvent prior to addition to the aqueous solution. The person skilled in the art is able to prepare aqueous solutions of amino acids or fatty acids having insufficient solubility by adding suitable organic and/or polar solvents. Such solvents are preferably provided in the form of an organic phase comprising liquid organic solvent. In a preferred embodiment, the organic solvent or phase is considered liquid when liquid at 25° C. and standard atmospheric pressure. In another preferred embodiment, a fatty acid is provided in the form of a fatty acid ester such as the respective methyl or ethyl ester. For example, the fatty acid laurate may be solved in lauric acid methyl ester as described in EP11191520.3. According to the present invention, the compounds and catalysts may be contacted in vitro, i.e. in a more or less enriched or even purified state, or may be contacted in situ, i.e. they are made as part of the metabolism of the cell and subsequently react inside the cell.

The term “an aqueous solution” comprises any solution comprising water, preferably mainly water as solvent that may be used to keep the inventive cell, at least temporarily, in a metabolically active and/or viable state and comprises, if such is necessary, any additional substrates. The person skilled in the art is familiar with the preparation of numerous aqueous solutions, usually referred to as media that may be used to keep inventive cells, for example LB medium in the case of E. coli. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium.

It is a particular strength of the present invention that not only saturated fatty acids, but also unsaturated fatty acids may be converted to acyl amino acids. In case the end product sought-after is to comprise a higher yield of saturated acyl residues than is present after step b), it may be possible to complement the process by hydrogenating the acyl residues of the acyl amino acids following step b). In this case, the inventive composition according to the fifth aspect of the present invention, which composition comprises a mixture of acyl amino acids having unsaturated acyl residues, constitutes an obligatory intermediate which may be converted to the final product, i.e. a mixture of acyl amino acids having saturated acyl residues. The hydrogenation may be carried out according to various state of the art processes, for example those described in U.S. Pat. No. 5,734,070. Briefly, the compound to be hydrogenated may be incubated at 100° C. in the presence of hydrogen and a suitable catalyst, for example a nickel catalyst on silicon oxide as a support.

The fatty acids that are to be converted to acyl amino acids may be produced by the cell according to the present invention. In one example, the very cell that produces the acyl amino acids is capable of producing the fatty acids from which the acyl amino acids are produced. In particular, the cells may be genetically modified to be able to produce fatty acids. In one example, the genetic modification may be to decrease a specific enzymatic activity and this may be done by a gene disruption or a genetic modification. The genetic modification may also increase a specific enzymatic activity. In particular, the genetic modification may increase microbial synthesis of a selected fatty acid or fatty acid derived chemical product above a rate of a control or wild type cell. This control or wild type cell may lack this genetic modification to produce a selected chemical product.

In particular, the cell comprises at least one genetic mutation that enables the cell to produce at least one fatty acid. In particular, the genetic mutation may enable the cell to produce at least one fatty acid by means of a malonyl-CoA dependent and malonyl-ACP independent fatty acyl-CoA metabolic pathway. More in particular, there is an increase in enzymatic activity in the malonyl-CoA dependent and malonyl-ACP independent fatty acyl-CoA metabolic pathway in the cell relative to the wild type cell. The cell may be genetically modified for increased enzymatic activity in the microorganism's malonyl-CoA dependent, malonyl-ACP independent, fatty acyl-CoA metabolic pathway (“MDMIFAA”) This pathway is also referred to herein as malonyl-CoA dependent, but malonyl-ACP independent, fatty acyl-CoA metabolic pathway. Such increase in the cell's malonyl-CoA dependent, malonyl-ACP independent fatty acyl-CoA metabolic pathway can be achieved by an increased activity or expression of a gene or a pathway comprising an acetoacetyl-CoA synthase, a ketoacyl-CoA synthase (or elongase), an enoyl-CoA reductase, a ketoacyl-CoA reductase and/or a 3-hydroxyacyl-CoA dehydratase in combination with a decrease in expression or activity of acetoacetyl-CoA thiolase. Alternatively, increased activity in the microorganism's malonyl-CoA dependent, malonyl-ACP independent fatty acyl-CoA metabolic pathway can be achieved by an increased expression of a gene or a pathway comprising an acetoacetyl-CoA synthase, a ketoacyl-CoA thiolase, a enoyl-CoA reductase, a ketoacyl-CoA reductase and/or a 3-hydroxyacyl-CoA dehydratase in combination with a decrease in expression or activity of acetoacetyl-CoA thiolase.

A list of non-limiting genetic modifications to enzymes or enzymatic activities that may lead a cell to produce a fatty acid and/or acyl coenzyme A thereof and that may be considered as at least one genetic mutation according to any aspect of the present invention is provided below in Table 1 and explained in US20140051136.

In one example, nucleic acid sequences that encode temperature-sensitive forms of these polypeptides may be introduced in place of the native enzymes, and when such genetically modified microorganisms are cultured at elevated temperatures (at which these thermolabile polypeptides become inactivated, partially or completely, due to alterations in protein structure or complete denaturation), there is observed an increase in a chemical product. For example, in E. coli, these temperature-sensitive mutant genes could include fabI^{ts}(S241F), fabB^{ts}(A329V) or fabD^{ts}(W257Q) amongst others. In most of these examples, the genetic modifications may increase malonyl-CoA utilization so that there is a reduced conversion of malonyl-CoA to fatty acids via the native pathway, overall biomass, and proportionally greater conversion of carbon source to a chemical product including a fatty acid or fatty acid derived product via a malonyl-CoA dependent and malonyl-ACP independent route. Also, additional genetic modifications, such as to increase malonyl-CoA production, may be made for some examples.

In another example, the enzyme, enoyl-acyl carrier protein reductase (EC No. 1.3.1.9, also referred to as enoyl-ACP reductase) is a key enzyme for fatty acid biosynthesis from malonyl-CoA. In Escherichia coli this enzyme, FabI, is encoded by the gene fabI (Richard J. Heath et al., 1995). In one example, the expression levels of a pyruvate oxidase gene (Chang et al., 1983, Abdel-Ahmid et al., 2001) can be reduced or functionally deleted in the cell according to any aspect of the present invention. The pyruvate oxidase gene may encode an enzyme of, for example, EC 1.2.3.3. In particular, the pyruvate oxidase gene may be a poxB gene. In one example, the expression levels of a lactate dehydrogenase gene (Mat-Jan et al., Bunch et al., 1997) can be reduced or functionally deleted. In some examples, the lactate dehydrogenase gene encodes an enzyme of, for example, EC 1.1.1.27. The lactate dehydrogenase gene may be an NAD-linked fermentative D-lactate dehydrogenase gene. In particular, the lactate dehydrogenase gene is a IdhA gene.

In one example, the genetic mutation that may increase the expression of fatty acids in the cell may be in at least one feedback resistant enzyme of the cell that results in increased expression of the feedback resistant enzyme. In particular, the enzyme may be pantothenate kinase, pyruvate dehydrogenase or the like. In E. coli, these feedback resistant mutant genes could include coaA(R106A) and/or Ipd(E354K).

In a further example, the increase in the cell's malonyl-CoA dependent, but malonyl-ACP independent fatty acyl-CoA metabolic pathway may occur through reduction in the acetoacetyl-CoA thiolase activity and/or trigger factor activity and/or in the activity of a molecular chaperone involved in cell division. In one example, the cell may comprise a genetic mutation in tig gene.

In one example, the genetic mutation in the cell may result in increased enzymatic activity in the NADPH-dependent transhydrogenase pathway relative to the wild type cell. This result may occur by introduction of a heterologous nucleic acid sequence coding for a polypeptide encoding nucleotide transhydrogenase activity. In another example, the genetic mutation in the cell may result in decreased expression of fatty acyl-CoA synthetase and/or ligase activity via any method known in the art. In yet another example, the genetic mutation in the cell may result in overexpression of an enzyme having acetyl-CoA carboxylase activity.

In one example, the cell may have increased intracellular bicarbonate levels brought about by introduction of a heterologous nucleic acid sequence coding for a polypeptide having cyanase and/or carbonic anhydrase activity. More in particular, the genetic mutation according to any aspect of the cell may result in increased and/or decreased levels of fatty acyl-CoA thioesterase activity. This result may increase chain length specificity of a desired fatty acid product by increasing levels of chain length specific fatty acyl-CoA thioesterase activity and decreasing the activity of fatty acyl-CoA thioesterase activity on undesired fatty acid chain lengths. In one example, the increased chain length specificity of fatty acid or fatty acid derived product may occur by increasing levels of chain length specific ketoacyl-CoA thiolase, enoyl-CoA reductase, ketoacyl-CoA reductase or 3-hydroxyacyl-CoA dehydratase activities either individually or in combination.

The genetic mutation in the cell according to any aspect of the present invention may result in an increase or decrease in expression of only one enzyme selected from the list of enzymes mentioned above or an increase or decrease in expression of a combination of enzymes mentioned above.

In another example, the genetic mutation in the cell may be in at least one enzyme selected from the group consisting of acetoacetyl-CoA synthase, ketoacyl-CoA synthase (or elongase), enoyl-CoA reductase, ketoacyl-CoA reductase, 3-hydroxyacyl-CoA dehydratase and acetoacetyl-CoA thiolase. More in particular, the genetic mutation in the cell may result in an increase in expression of acetoacetyl-CoA synthase, ketoacyl-CoA synthase (or elongase), enoyl-CoA reductase, ketoacyl-CoA reductase and 3-hydroxyacyl-CoA dehydratase in combination optionally with a decrease in expression or activity of acetoacetyl-CoA thiolase. In particular, the enoyl-CoA reductase and/or ketoacyl-CoA reductase may either utilize the cofactor NADH and/or NADPH.

In particular, the genetic modification in the cell according to any aspect of the present invention may comprise any of the enzymes listed in Table 1 in combination with the following enzymes acetoacetyl-CoA synthase, ketoacyl-CoA synthase (or elongase), enoyl-CoA reductase, ketoacyl-CoA reductase and/or 3-hydroxyacyl-CoA dehydratase and acetoacetyl-CoA thiolase wherein the expression or activity of enzymes acetoacetyl-CoA synthase, ketoacyl-CoA synthase (or elongase), enoyl-CoA reductase, ketoacyl-CoA reductase and 3-hydroxyacyl-CoA dehydratase is increased and the activity of acetoacetyl-CoA thiolase is decreased.

In yet another example, malonyl-CoA dependent, malonyl-ACP independent fatty acyl-CoA metabolic pathway in the cell according to any aspect of the present invention can be achieved by an increased expression of a gene or a pathway comprising acetoacetyl-CoA synthase, ketoacyl-CoA thiolase, enoyl-CoA reductase, ketoacyl-CoA reductase and/or 3-hydroxyacyl-CoA dehydratase in combination with a decrease in expression or activity of acetoacetyl-CoA thiolase.

In particular, the genetic modification in the cell according to any aspect of the present invention may comprise any of the enzymes listed in Table 1 in combination with the following enzymes acetoacetyl-CoA synthase, ketoacyl-CoA thiolase, enoyl-CoA reductase, ketoacyl-CoA reductase and/or 3-hydroxyacyl-CoA dehydratase in combination with a decrease in expression or activity of acetoacetyl-CoA thiolase.

In one example, the cell according to any aspect of the present invention may comprise a genetic modification in any of the enzymes listed in Table 1 in combination with the following enzymes acetyl-CoA carboxylase, malonyl-CoA:ACP transacylase (FabD), β-ketoacyl-ACP synthase III, β-ketoacyl-ACP synthase I (FabB), β-ketoacyl-ACP synthase II (FabF), 3-oxoacyl-ACP-synthase I and enoyl ACP reductase.

More in particular, the genetic mutation may result in an increase in the expression of at least one enzyme selected from the group consisting of acetyl-CoA carboxylase, malonyl-CoA:ACP transacylase (FabD), β-ketoacyl-ACP synthase III, β-ketoacyl-ACP synthase I (FabB), β-ketoacyl-ACP synthase II (FabF), 3-oxoacyl-ACP-synthase I and enoyl ACP reductase relative to the wild type cell. In particular, the genetic mutation may result in an increase in the expression of more than one enzyme in the cell according to any aspect of the present invention that enables the cell to produce a fatty acid and/or acyl coenzyme A thereof by means of increased enzymatic activity in the cell relative to the wild type cell of malonyl-CoA dependent and malonyl-ACP independent fatty acyl-CoA metabolic pathway.

In one example, there may be an increase in expression of β-ketoacyl-ACP synthase and 3-oxoacyl-ACP-synthase in the cell according to any aspect of the present invention. In another example, there may be an increase in expression of β-ketoacyl-ACP synthase and Malonyl-CoA-ACP transacylase in the cell according to any aspect of the present invention. In yet another example, there may be an increase in expression of β-ketoacyl-ACP synthase and enoyl ACP reductase in the cell according to any aspect of the present invention. In one example, there may be an increase in expression of β-ketoacyl-ACP synthase, Malonyl-CoA-ACP transacylase and enoyl ACP reductase in the cell according to any aspect of the present invention. In all examples, there may be an increase in the expression of enoyl ACP reductase and/or acyl-CoA thioesterase.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that code for a corresponding enzyme with increased activity and optionally by combining these measures. Genetically modified cells used according to any aspect of the present invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extra-chromosomally replicating vector.

In one example, the genetic modification in a cell of the present invention may include any of the enzymes above in combination with a genetic modification in at least one enzyme selected from the group consisting of β-ketoacyl-ACP synthase I, 3-oxoacyl-ACP-synthase I, Malonyl-CoA-ACP transacylase, enoyl ACP reductase, and β-ketoacyl-ACP synthase III. In particular, the genetic modification in the cell according to any aspect of the present invention may comprise any of the enzymes listed in Table 1 in combination with the following enzymes β-ketoacyl-ACP synthase I, 3-oxoacyl-ACP-synthase I, Malonyl-CoA-ACP transacylase, enoyl ACP reductase, and β-ketoacyl-ACP synthase III.

TABLE 1
Examples of genetic modifications in cells of microorganisms for production of fatty acids
Genetic Modifications

| | E.C. CLASSIFI- | GENE NAME | |
|---|---|---|---|
| ENZYME FUNCTION | CATION No. | IN E. COLI | COMMENTS |
| Glucose transporter | N/A | galP | Increase function |
| Pyruvate dehydrogenase E1p | 1.2.4.1 | aceE | Increase function |
| lipoate acetyltransferase/ | 2.3.1.12 | aceF | Increase function |
| dihydrolipoamide acetyltransferase | | | |
| Pyruvate dehydrogenase | 1.8.1.4 | lpd | Increase function or |
| E3 (lipoamide dehydrogenase) | | | alter such as by |
| | | | mutation to increase |
| | | | resistance to NADH |
| | | | inhibition, |
| Lactate dehydrogenase | 1.1.1.28 | ldhA | Decrease function, |
| | | | including by mutation |
| Pyruvate formate lyase | 2.3.1.— | pflB | Decrease function, |
| (B “inactive”) | | | including by mutation |
| Pyruvate oxidase | 1.2.2.2 | poxB | Decrease function, |
| | | | including by mutation |
| Phosphate acetyltransferase | 2.3.1.8 | Pta | Decrease function, |
| | | | including by mutation |
| acetate kinase | 2.7.2.15 2.7.2.1 | ackA | Decrease function, |
| | | | including by mutation |
| methylglyoxal synthase | 4.2.3.3 | mgsA | Decrease function, |
| | | | including by mutation |
| Heat stable, histidyl | N/A | ptsH | Decrease function, |
| phosphorylatable protein (of PTS) | | (HPr) | including by mutation |
| Phosphoryl transfer protein | N/A | ptsI | Decrease function, |
| (of PTS) | | | including by mutation |
| Polypeptide chain (of PTS) | N/A | Crr | Decrease function, |
| | | | including by mutation |
| 3-oxoacyl-ACP synthase I | 2.3.1.179 | fabF | Decrease function, |
| 3-oxoacyl-ACP synthase II | 2.3.1.41 | | including by mutation |
| monomer | | | |
| β-ketoacyl-ACP synthase I, | 2.3.1.41 | fabB | Decrease function, |
| 3-oxoacyl-ACP-synthase I | 2.3.1.— | | including by mutation |
| Malonyl-CoA-ACP | 2.3.1.39 | fabD | Decrease function, |
| transacylase | | | including by mutation |
| enoyl acyl carrier protein | 1.3.1.9, | fabI | Decrease function, |
| reductase | 1.3.1.10 | | including by mutation |
| β-ketoacyl-acyl carrier | 2.3.1.180 | fabH | Decrease function, |
| protein synthase III | | | including by mutation |
| Carboxyl transferase | 6.4.1.2 | accA | Increase function |
| subunit α subunit | | | |
| Biotin carboxyl carrier | 6.4.1.2 | accB | Increase function |
| protein | | | |
| Biotin carboxylase subunit | 6.3.4.14 | accC | Increase function |
| Carboxyl transferase | 6.4.1.2 | accD | Increase function |
| subunit β subunit | | | |
| long chain fatty acyl | 3.1.2.2, | tesA | Increase function as |
| thioesterase I | 3.1.1.5 | | well as alter by |
| | | | mutation to express in |
| | | | cytoplasm or deletion |
| acyl-CoA synthase | 2.3.1.86 | fadD | Decrease via deletion |
| | | | or mutation |
| acetate CoA-transferase | 2.8.3.8 | atoD | Decrease via deletion |
| | | | or mutation |
| acetate CoA-transferase | 2.8.3.8 | atoA | Decrease via deletion |
| | | | or mutation |
| Transporter | N/A | atoE | Decrease via deletion |
| | | | or mutation |
| acetyl-CoA acetyltransferase | 2.3.1.9 | atoB | Decrease via deletion |
| | | | or mutation |
| pantothenate kinase | 2.7.1.33 | coaA | Increase via expression |
| | | | or feedback resistant |
| | | | mutation |
| lactose repressor | N/A | lacI | Decrease via deletion |
| | | | or mutation |
| γ-glutamyl-γ- | 1.2.1.— | puuC | Decrease via deletion |
| aminobutyraldehyde | | | or mutation |
| dehydrogenase | | | |
| malate synthase A | 2.3.3.9 | aceB | Decrease via deletion |
| | | | or mutation |
| isocitrate lyase | 4.1.3.1 | aceA | Decrease via deletion |
| | | | or mutation |
| isocitrate dehydrogenase | 3.1.3.— | aceK | Decrease via deletion |
| phosphatase/isocitrate | 2.7.11.5. | | or mutation |
| dehydrogenase kinase | | | |
| pyruvate formate-lyase | 1.2.1.10 1.1.1.1 | adhE | Decrease via deletion |
| deactivase | | | or mutation |
| aldehyde dehydrogenase A, | 1.2.1.21 1.2.1.22 | aldA | Decrease via deletion |
| NAD-linked | | | or mutation |
| acetaldehyde | 1.2.1.4 | aldB | Decrease via deletion |
| dehydrogenase | | | or mutation |
| Lambda phage DE3 lysogen | N/A | λDE3 | Increase |
| T7 mRNA polymerase | N/A | T7pol | Increase |
| trigger factor | 5.2.1.8 | tig | Decrease via deletion |
| | | | or mutation |
| 3-ketoacyl-CoA thiolase | 2.3.1.16 | fadA | Increase |
| dodecenoyl-CoA δ-isomerase, | 5.3.3.8 1.1.1.35 | fadB | Increase |
| enoyl-CoA hydratase, | 5.1.2.3 4.2.1.17 | | |
| 3-hydroxybutyryl-CoA epimerase, | | | |
| 3-hydroxyacyl-CoA dehydrogenase | | | |
| Sucrose permease | N/A | cscB | Increase |
| Invertase | 3.2.1.26 | cscA | Increase |
| fructokinase | 2.7.1.4 | cscK | Increase |
| carbonic anhydrase | 4.2.1.1 | cynT | Increase |
| carbonic anhydrase | 4.2.1.1 | can | Increase |
| pyridine nucleotide | 1.6.1.2 | pntAB | Increase |
| transhydrogenase | | | |
| pyridine nucleotide | 1.6.1.1 | udhA | Increase |
| transhydrogenase | | | |
| acyl-CoA thioesterase | 3.1.2.20 3.1.2.2 | yciA | Increase and or |
| | | | decrease |
| thioesterase II | 3.1.2.20 3.1.2.2 | tesB | Increase and or |
| | | | decrease |
| thioesterase III | 3.1.2.— | fadM | Increase and or |
| | | | decrease |
| hydroxyphenylacetyl-CoA | N/A | paaI | Increase and or |
| thioesterase | | | decrease |
| esterase/thioesterase | 3.1.2.28 | ybgC | Increase and or |
| | | | decrease |
| proofreading thioesterase in | | entH | Increase and or |
| enterobactin biosynthesis | | | decrease |
| acetoacetyl-CoA synthase | 2.3.1.194 | npth07 | Increase |
| 3-ketoacyl-CoA synthase/elongase | 2.3.1 | Elo1 | Increase |
| 3-ketoacyl-CoA synthase/elongase | 2.3.1 | Elo2 | Increase |
| 3-Hydroxybutyryl-CoA dehydrogenase | 1.1.1.157 | hbd | Increase |
| 3-oxoacyl-CoA reductase | 1.1.1.100 | fabG | Increase |
| enoyl-CoA hydratase | 4.2.1.17 | crt | Increase |
| enoyl-CoA hydratase | 4.2.1.17 | ech2 | Increase |
| Trans-2-enoyl-reductase | 1.3.1.9 | ter | Increase |
| thioesterase | 3.1.2.20 | paaI | Decrease |

E.C. No. = “Enzyme Commission number”

According to any aspect of the present invention, the cell may be genetically modified to increase the expression of at least one transporter protein compared to the wild type cell. The transporter protein may be FadL and/or AlkL. In one example, the cell may be genetically modified to overexpress both the FadL and the AlkL gene.

The cell may also be genetically modified to increase the expression of at least one enzyme selected from the group consisting of acetoacetyl-CoA synthase, ketoacyl-CoA synthase (or elongase), ketoacyl-CoA thiolase, enoyl-CoA reductase, ketoacyl-CoA reductase and 3-hydroxyacyl-CoA dehydratase and optionally a decrease in in the expression of acetoacetyl-CoA thiolase relative to the wild type cell wherein the cell has a reduced fatty acid degradation capacity.

Accordingly, the cells and methods of the present invention may comprise providing a genetically modified microorganism that comprises both a production pathway to a fatty acid or fatty acid derived product, and a modified polynucleotide that encodes an enzyme of the malonyl-ACP dependent fatty acid synthase system that exhibits reduced activity, so that utilization of malonyl-CoA shifts toward the production pathway compared with a comparable (control) microorganism lacking such modifications. The methods involve producing the chemical product using a population of such genetically modified microorganism in a vessel, provided with a nutrient media. Other genetic modifications described herein, to other enzymes, such as acetyl-CoA carboxylase and/or NADPH-dependent transhydrogenase, may be present in some such examples. Providing additional copies of polynucleotides that encode polypeptides exhibiting these enzymatic activities is shown to increase a fatty acid or fatty acid derived product production. Other ways to increase these respective enzymatic activities is known in the art and may be applied to various examples of the present invention.

Also, without being limiting, a first step in some multi-phase method embodiments of making a fatty acid may be exemplified by providing into a vessel, such as a culture or bioreactor vessel, a nutrient media, such as a minimal media as known to those skilled in the art, and an inoculum of a genetically modified microorganism so as to provide a population of such microorganism, such as a bacterium, and more particularly a member of the family Enterobacteriaceae, such as E. coli, where the genetically modified microorganism comprises a metabolic pathway that converts malonyl-CoA to a fatty acid. This inoculum is cultured in the vessel so that the cell density increases to a cell density suitable for reaching a production level of a fatty acid or fatty acid derived product that meets overall productivity metrics taking into consideration the next step of the method. In various alternative embodiments, a population of these genetically modified microorganisms may be cultured to a first cell density in a first, preparatory vessel, and then transferred to the noted vessel so as to provide the selected cell density. Numerous multi-vessel culturing strategies are known to those skilled in the art. Any such embodiments provide the selected cell density according to the first noted step of the method.

Also without being limiting, a subsequent step may be exemplified by two approaches, which also may be practiced in combination in various embodiments. A first approach provides a genetic modification to the genetically modified microorganism such that its enoyl-ACP reductase enzymatic activity may be controlled. As one example, a genetic modification may be made to substitute a temperature-sensitive mutant enoyl-ACP reductase (e.g., fabI^{TS} in E. coli) for the native enoyl-ACP reductase. The former may exhibit reduced enzymatic activity at temperatures above 30° C. but normal enzymatic activity at 30° C., so that elevating the culture temperature to, for example to 34° C., 35° C., 36° C., 37° C. or even 42° C., reduces enzymatic activity of enoyl-ACP reductase. In such case, more malonyl-CoA is converted to a fatty acid or fatty acid derived product or another chemical product than at 30° C., where conversion of malonyl-CoA to fatty acids is not impeded by a less effective enoyl-ACP reductase.

Other genetic modifications that may be useful in the production of fatty acids and/or amino acids may be included in the cell. For example, the ability to utilise sucrose may be provided, and this would expand the range of feed stocks that can be utilised to produce a fatty acid or fatty acid derived product or other chemical products. Common laboratory and industrial strains of E. coli, such as the strains described herein, are not capable of utilizing sucrose as the sole carbon source. Since sucrose, and sucrose-containing feed stocks such as molasses, are abundant and often used as feed stocks for the production by microbial fermentation, adding appropriate genetic modifications to permit uptake and use of sucrose may be practiced in strains having other features as provided herein. Various sucrose uptake and metabolism systems are known in the art (for example, U.S. Pat. No. 6,960,455).

Also, genetic modifications may be provided to add functionality for breakdown of more complex carbon sources, such as cellulosic biomass or products thereof, for uptake, and/or for utilisation of such carbon sources. For example, numerous cellulases and cellulase-based cellulose degradation systems have been studied and characterized (Beguin, P and Aubert, J-P (1994) FEMS Microbial. Rev. 13: 25-58; Ohima, K. et al. (1997) Biotechnol. Genet. Eng. Rev. 14: 365414).

In some examples, genetic modifications increase the pool and availability of the cofactor NADPH, and/or, consequently, the NADPH/NADP⁺ ratio may also be provided. For example, in E. coli, this may be done by increasing activity, such as by genetic modification, of one or more of the following genes: pgi (in a mutated form), pntAB, overexpressed, gapA:gapN substitution/replacement, and disrupting or modifying a soluble transhydrogenase such as sthA, and/or genetic modifications of one or more of zwf, gnd, and edd.

Any such genetic modifications may be provided to species not having such functionality, or having a less than desired level of such functionality. More generally, and depending on the particular metabolic pathways of a microorganism selected for genetic modification, any subgroup of genetic modifications may be made to decrease cellular production of fermentation product(s) selected from the group consisting of acetate, acetoin, acetone, acrylic, malate, Benzoyl-CoA, fatty acid ethyl esters, isoprenoids, glycerol, ethylene glycol, ethylene, propylene, butylene, isobutylene, ethyl acetate, vinyl acetate, other acetates, 1,4-butanediol, 2,3-butanediol, butanol, isobutanol, sec-butanol, butyrate, isobutyrate, 2-OH-isobutryate, 3-OH-butyrate, ethanol, isopropanol, D-lactate, L-lactate, pyruvate, itaconate, levulinate, glucarate, glutarate, caprolactam, adipic acid, propanol, isopropanol, fusel alcohols, and 1,2-propanediol, 1,3-propanediol, formate, fumaric acid, propionic acid, succinic acid, valeric acid, and maleic acid. Gene deletions may be made as disclosed generally herein, and other approaches may also be used to achieve a desired decreased cellular production of selected fermentation products.

The inventions is further illustrated by the following figures and non-limiting examples from which further embodiments, aspects and advantages of the present invention may be taken.

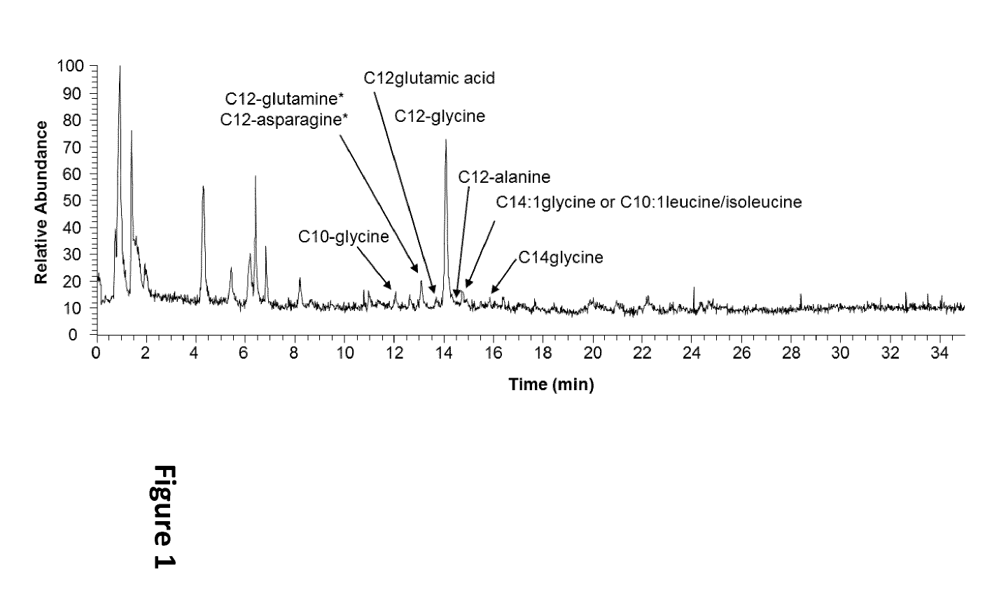

FIG. 1 depicts a total ion chromatogram of the 48 h sample from the E. coli W3110 ΔfadE pJ294{Ptac}[synUcTE]/pCDF{Ptac}[hGLYAT2(co_Ec)_fadD_Ec] fermentation.

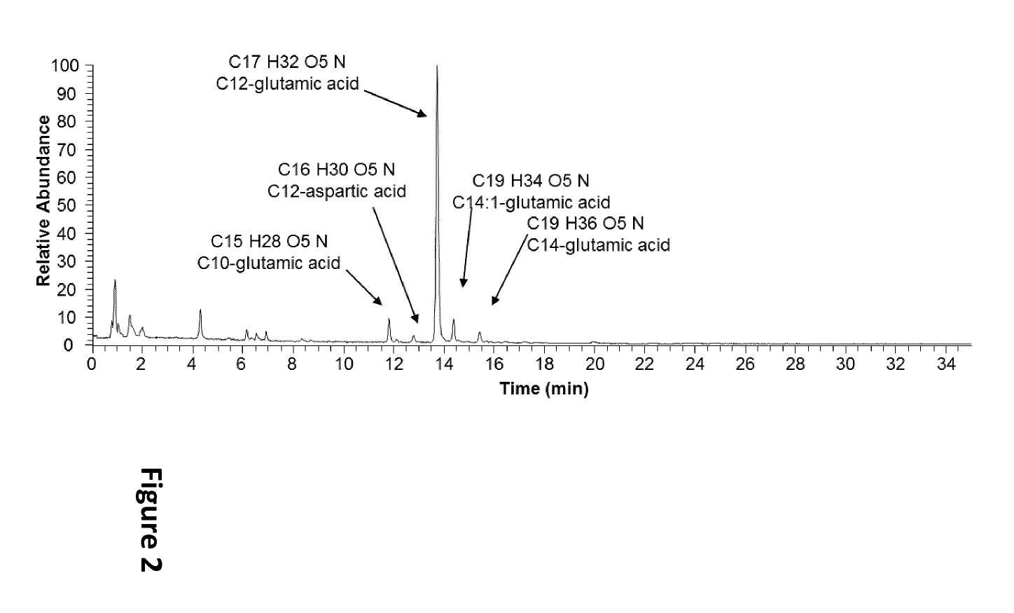

FIG. 2 depicts a total ion chromatogram of the 48 h sample from the E. coli W3110 ΔfadE pJ294{Ptac}[synUcTE]/pCDF{Ptac}[hGLYAT3(co_Ec)_fadD_Ec] fermentation.

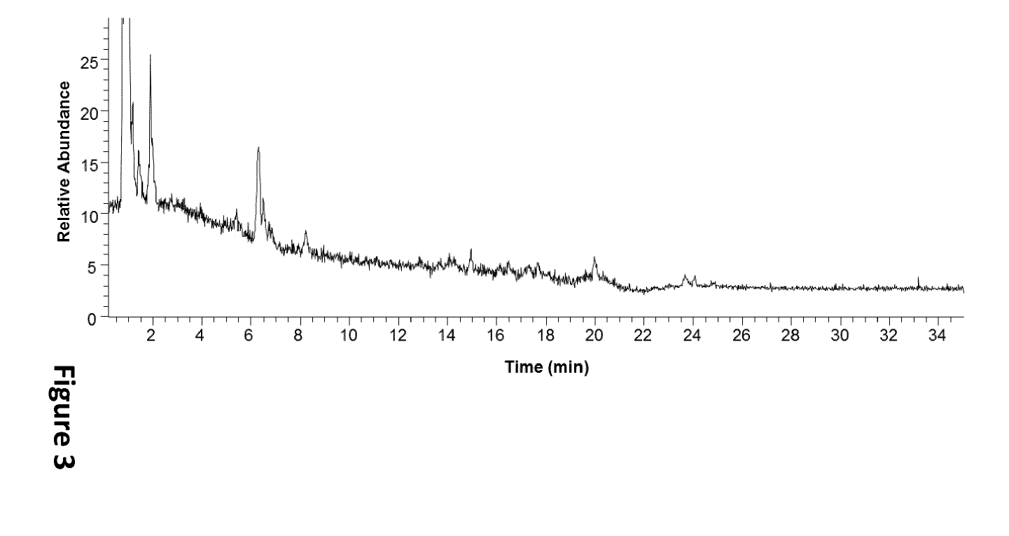

FIG. 3 depicts a total ion chromatogram of the 48 h sample from the E. coli W3110 ΔfadE pJ294{Ptac}[synUcTE]/pCDFDuet-1 fermentation (negative control).

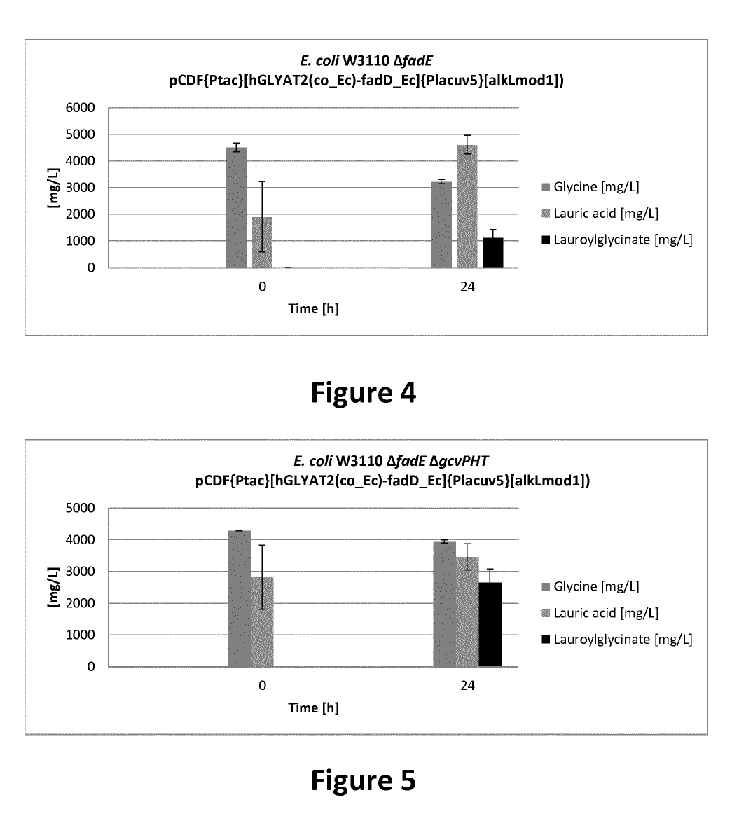

FIG. 4 depicts production of lauroylglycinate by E. coli strains W3110 ΔfadE pCDF{Ptac}[hGLYAT2(co_Ec)-fadD_Ec]{Placuv5}[alkLmod1].

FIG. 5 depicts production of lauroylglycinate by E. coli strain W3110 ΔfadE ΔgcvTHP pCDF{Ptac}[hGLYAT2(co_Ec)-fadD_Ec]{Placuv5}[alkLmod1].

FIG. 6 A tree showing the percentage sequence identity of GLYAT2 in the various organisms tested in Example 16.

### EXAMPLES

### Sequence ID NOs

Throughout this application a range of SEQ ID NOs are used. These are shown in Table 2 below.

TABLE 2
Sequences used in the examples.

| SEQ ID | |
|---|---|
| NO: | Comment |
| 1 | Umbellularia californica synUcTE (an acyl-CoA thioesterase) |
| | gene (codon-optimized) |
| 2 | tac promoter |
| 3 | Vector pJ294[Ptac-synUcTE], see example 1 |
| 4 | Homo sapiens genes hGLYAT2 (an amino acid N-acyl |
| | transferase) |
| 5 | Homo sapiens genes hGLYAT3 (another amino acid N-acyl |
| | transferase) |
| 6 | Escherichia coli fadD (an acyl-CoA synthetase) |
| 7 | Vector pCDF[atfA1_Ab(co_Ec)-fadD_Ec], see example 2 |
| 8 | Vector pCDF{Ptac}[hGLYAT2(co_Ec)-fadD_Ec], see example 2 |
| 9 | Vector pCDF{Ptac}[hGLYAT3(co_Ec)-fadD_Ec], see example 2 |
| 10 | alkL (an importer facilitating transport of hydrophobic |
| | acyl across cell membranes) gene, see example 3 |
| 11 | lacuv5 promoter, see example 3 |
| 12 | Vector pCDF[alkLmod1], see example 3 |
| 13 | Vector pCDF{Ptac}[hGLYAT2(co_Ec)-fadD_Ec]{Placuv5}[alkLmod1], |
| | see example 3 |
| 14 | Vector pET-28b, see example 10 |
| 15 | Vector pET-28b{Ptac}[hGLYAT2(co_Ec)], see example 10 |
| 16 | pET-28b{Ptac}[hGLYAT3(co_Ec)], see example 10 |

### Example 1

### Generation of an Expression Vector for the Umbellularia californica Gene synUcTE

To generate an expression vector for the Umbellularia californica synUcTE gene (SEQ ID NO: 1), which encodes the Umbellularia californica acyl CoA-thioesterase, this gene was codon-optimized for expression in Escherichia coli. The gene was synthesized together with a tac promoter (SEQ ID NO: 2), and, simultaneously, one cleavage site was introduced upstream of the promoter and one cleavage site downstream of the terminator. The synthesized DNA fragment P_{tac}-synUcTE was digested with the restriction endonucleases BamHI and NotI and ligated into the correspondingly cut vector pJ294 (DNA2.0 Inc., Menlo Park, Calif., USA). The finished E. coli expression vector was referred to as pJ294[Ptac-synUcTE] (SEQ ID NO: 3).

### Example 2

### Generation of Vectors for Coexpression of Escherichia coli fadD with Either the Homo sapiens Genes hGLYAT3 and hGLYAT2

To generate vectors for the coexpression of the Homo sapiens genes hGLYAT2 (SEQ ID NO: 4) or hGYLAT3 (SEQ ID NO: 5), which encodes human glycine-N-acyltransferase, with Escherichia coli fadD (SEQ ID NO: 6), which encodes the E. coli acyl-CoA synthetase, the genes hGLYAT2 and hGLYAT3 were codon-optimized for expression in Escherichia coli and synthesized. The synthesized DNA fragments were digested with the restriction endonucleases SacII and Eco47III and ligated into the correspondingly cut pCDF[atfA1_Ab(co_Ec)-fadD_Ec] (SEQ ID NO: 7) with removal of the aftA1 gene. The sequence segments which were additionally removed in this process were cosynthesized during gene synthesis. The vector is a pCDF derivative which already comprises a synthetic tac promoter (SEQ ID NO: 2) and the Escherichia coli fadD gene. The resulting expression vectors were named pCDF{Ptac}[hGLYAT2(co_Ec)-fadD_Ec] (SEQ ID NO: 8) and pCDF{Ptac}[hGLYAT3(co_Ec)-fadD_Ec] (SEQ ID NO: 9).

### Example 3

### Generation of Vectors for the Coexpression of the Homo sapiens hGLYAT2, Escherichia coli fadD and Pseudomonas putida alkL Genes

To generate vectors for the coexpression of the hGLYAT2 genes with a modified Pseudomonas putida alkL gene, which encodes AlkL, an outer membrane protein that facilitates the import of hydrophobic substrates into a cell, the alkL gene (SEQ ID NO: 10) was amplified together with the lacuv5 promoter (SEQ ID NO: 11) from the plasmid pCDF[alkLmod1] (SEQ ID NO: 12) by means of sequence-specific oligonucleotides. The PCR products were cleaved with the restriction endonucleases BamHI and NsiI and ligated into the correspondingly cleaved vector pCDF{Ptac}[hGLYAT2(co_Ec)-fadD_Ec] (SEQ ID NO: 8). The correct insertion of the target genes was checked by restriction analysis and the authenticity of the introduced genes was verified by DNA sequencing. The resulting expression vector was named pCDF{Ptac}[hGLYAT2(co_Ec)-fadD_Ec]{Placuv5}[alkLmod1] (SEQ ID NO: 13).

The following parameters were used for PCR: 1×: initial denaturation, 98° C., 3:00 min; 35×denaturation, 98° C., 0:10 min; annealing, 65° C., 0:20 min; elongation, 72° C., 0:17 min; 1×: final elongation, 72° C., 10 min. For amplification the Phusion™ High-Fidelity Master Mix from New England Biolabs (Frankfurt) was used according to manufacturer's manual. 50 μL of the PCR reaction were analyzed on a 1% TAE agarose gel. Procedure of PCR, agarose gel electrophoresis, ethidium bromide staining of DNA and determination of PCR fragment size were carried out known to those skilled in the art

### Example 4

### Generation of an E. coli Strain with Deletion in the fadE Gene, which Strain Overexpresses the Umbellularia californica synUcTE, Escherichia coli fadD and Homo sapiens hGLYAT2 and hGLYAT3 Genes

To generate E. coli strains which coexpress the Umbellularia californica synUcTE in combination with the Escherichia coli fadD and Homo sapiens hGLYAT2 or Homo sapiens hGLYAT3 genes, the strain E. coli W3110 ΔfadE was transformed with the plasmids pJ294{Ptac}[synUcTE] and pCDF{Ptac}[hGLYAT2(co_Ec)_fadD_Ec] or pCDF{Ptac}[hGLYAT3(co_Ec)_fadD_Ec] by means of electroporation and plated onto LB-agar plates supplemented with spectinomycin (100 μg/mL) and ampicillin (100 μg/mL). Transformants were checked for the presence of the correct plasmids by plasmid preparation and analytic restriction analysis. The strains E. coli W3110 ΔfadE pJ294{Ptac}[synUcTE]/pCDF{Ptac}[h GLYAT2(co_Ec)_fadD_Ec] and E. coli W3110 ΔfadE pJ294{Ptac}[synUcTE]/pCDF{Ptac}[h GLYAT3(co_Ec)_fadD_Ec] were generated thus.

### Example 5

### Generation of an E. coli Strain with Deletion in the fadE Gene, which Strain Overexpresses the Escherichia coli fadD and Either Homo sapiens hGLYAT2 or hGLYAT3 Genes

To generate E. coli strains which overexpress the Escherichia coli fadD gene in combination with the Homo sapiens hGLYAT2 or hGLYAT3 genes, electrocompetent cells of E. coli strain W3110 ΔfadE were generated. E. coli W3110 ΔfadE was transformed with the plasmid pCDF{Ptac}[hGLYAT2(co_Ec)-fadD_Ec]{Placuv5}[alkLmod1] and plated onto LB-agar plates supplemented with spectinomycin (100 μg/mL). Transformants were checked for the presence of the correct plasmids by plasmid preparation and analytic restriction analysis. The strain generated thus was named E. coli W3110 ΔfadE pCDF{Ptac}[hGLYAT2(co_Ec)-fadD_Ec]{Placuv5}[alkLmod1].

### Example 6

### Production of Fatty Acid/Amino Acid Adducts by E. coli Strains with Deletion in the fadE Gene, which Strains Overexpress the synUcTE and fadD Genes in Combination with Either hGLYAT2 or hGLYAT3

The strains generated in Example 4 were used to study their ability to produce fatty acid/amino acid adducts, proceeding as follows:

Starting from a −80° C. glycerol culture, the strains to be studied were first plated onto an LB-agar plate supplemented with 100 μg/mL ampicillin and 100 μg/mL spectinomycin and incubated overnight at 37° C. Starting from a single colony in each case, the strains were then grown as a 5-mL preculture in Luria-Bertani broth, Miller (Merck, Darmstadt) supplemented with 100 μg/mL ampicillin and 100 μg/mL spectinomycin. The further culture steps were performed in M9 medium. The medium, composed of 38 mM disodium hydrogenphosphate dihydrate, 22 mM potassium dihydrogenphosphate, 8.6 mM sodium chloride, 37 mM ammonium chloride, 2% (w/v) glucose, 2 mM magnesium sulphate heptahydrate (all chemicals from Merck, Darmstadt) and 0.1% (v/v) trace element solution, was brought to pH 7.4 with 25% strength ammonium hydroxide solution. The trace element solution added, composed of 9.7 mM manganese(II) chloride tetrahydrate, 6.5 mM zinc sulphate heptahydrate, 2.5 mM sodium-EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate, dissolved in 1 M hydrochloric acid (all chemicals from Merck, Darmstadt) was filter-sterilized before being added to the M9 medium. 20 mL of M9 medium supplemented with 100 μg/mL spectinomycin and 100 μg/mL ampicillin were introduced into baffled 100-mL Erlenmeyer flasks and inoculated with 0.5 mL preculture. The flasks were cultured at 37° C. and 200 rpm in a shaker-incubator. After a culture time of 8 hours, 50 mL of M9 medium supplemented with 100 μg/mL spectinomycin and 100 μg/mL ampicillin were introduced into a baffled 250-mL Erlenmeyer flask and inoculated with the 10-mL culture to achieve an optical density (600 nm) of 0.2. The flasks were cultured at 37° C. and 200 rpm in a shaker-incubator. When an optical density (600 nm) of 0.7 to 0.8 was reached, gene expression was induced by addition of 1 mM IPTG. The strains were cultured for a further 48 hours at 30° C. and 200 rpm. Simultaneously with the induction, 1 g/L glycine was added to some of the cultures. During culturing, samples were taken, and fatty acid/amino acid adducts present were analysed. The results are shown in Figures. 1 and 2. It has been possible to demonstrate that both E. coli strain W3110 ΔfadE pJ294{Ptac}[synUcTE]/pCDF{Ptac}[hGLYAT2(co_Ec)_fadD_Ec] and E. coli strain W3110 ΔfadE pJ294{Ptac}[synUcTE]/pCDF{Ptac}[hGLYAT3(co_Ec)_fadD_Ec] are capable of forming various fatty acid/amino acid adducts, for example lauroyl-glutamic acid, from glucose. By contrast, no such adducts can be found in a cell that, as a negative control, lacks the plasmids (FIG. 3). It appears that slight sequence variations, for example amino acid substitutions that distinguish hGLYAT2 from hGLYAT3, do not compromise the ability of the cell to make the fatty acid/acyl amino acid adducts as shown in Table 3.

TABLE 3
Quantitative determination of fatty acid glycinates after 48 h culture time

| | C_{lauroylglycinate} | C_{myristoylglycinate} |
|---|---|---|
| Strain | [mg/L] | [mg/L] |
| E. coli W3110 ΔfadE pJ294{Ptac}[synUcTE]/ | 111 | <2 |
| pCDF{Ptac}[hGLYAT2(co_Ec)_fadD_Ec] | | |
| E. coli W3110 ΔfadE pJ294{Ptac}[synUcTE]/ | 121 | 2.8 |
| pCDF{Ptac}[hGLYAT2(co_Ec)_fadD_Ec] + | | |
| 1 g/L glycine | | |
| E. coli W3110 ΔfadE pJ294{Ptac}[synUcTE]/ | n.d. | n.d. |
| pCDFDuet-1 | | |

### Example 7

### Chromatographic Quantification of Products by HPLC/MS

The quantification of N-lauroylglycine, N-myristoylglycine and N-palmitoylglycine and the detection of other acylamino acids in fermentation samples was performed by HPLC-ESI/MS. The quantification was performed with the aid of an external calibration (approx. 0.1-50 mg/I) for the three target compounds in the “single ion monitoring” mode (SIM). In parallel, a scan was carried out over a mass range m/z=100-1000 so as to identify further acylamino acids.

The samples for the determination of the fatty acid glycinates were prepared as follows: 800 μL of solvent (acetone) and 200 μL of sample were pipetted into a 2-mL reaction vessel. The mixture was shaken in a Retsch mill for 1 minute at 30 Hz and then centrifuged for 5 min at approximately 13 000 rpm. The clear supernatant was removed using a pipette and, after suitable dilution with diluent (80% acetonitrile/20% water+0.1% formic acid), analyzed. The calibration standards used were likewise dissolved and diluted in this diluent.

The following equipment was employed:
Surveyor HPLC system (Thermo Scientific, Waltham, Mass., USA) composed of MS pump, Autosampler Plus and PDA Detector Plus
Mass spectrometer TSQ Vantage with HESI II source (Thermo Scientific, Waltham, Mass., USA)
HPLC column: 100×2 mm Pursuit XRS Ultra C8; 2.8 μm (Agilent, Santa Clara, Calif., USA)

Chemicals:
Water from a Millipore system
Acetonitrile for HPLC (Merck AG, Darmstadt, Germany)
Formic acid, p.a. grade (Merck, Darmstadt, Germany)
N-propanol Lichrosolv (Merck, Darmstadt, Germany)
N-lauroylglycine 99% (Chem-Impex International, Wood Dale, Ill., USA)
N-myristoylglycine >98% (Santa Cruz Biotechnology, Texas, USA)
N-palmitoylglycine >99% (provenance unknown)

The HPLC separation was carried out using the abovementioned HPLC column. The injection volume amounted to 2 μL, the column temperature to 40° C., the flow rate to 0.3 mL/min. The mobile phase consisted of Eluent A (0.1% strength (v/v) aqueous formic acid) and Eluent B (75% acetonitrile/25% n-propanol (v/v) with 0.1% (v/v) formic acid). The following gradient profile was used:

TABLE 4
Gradient profile used in Example 7.

| Time | Eluent A | Eluent B |
|---|---|---|
| [min] | [%] | [%] |
| 0 | 90 | 10 |
| 1 | 90 | 10 |
| 20 | 5 | 95 |
| 25 | 5 | 95 |

The HPLC/MS analysis was carried out under positive ionization mode with the following parameters of the ESI source:

Spray Voltage: 3500V

Vaporizer Temperature: 50° C.

Sheath Gas Pressure: 40

Aux. Gas Pressure: 10

Capillary Temperature: 250° C.

Sprayer Distance: Ring C

Detection and quantification of the three analytes were performed by “single ion monitoring”

(SIM) with the following parameters shown in Table 5.

TABLE 5
Parameters used in SIM of Example 7.

| | Ion [M + H] | Scan range | Scan time | Resolution |
|---|---|---|---|---|
| Analyte | [m/z] | [m/z] | [ms] | Q3 |
| N-lauroylglycine | 258.2 | 0.002 | 50 | 0.7 |
| N-myristoylglycine | 286.2 | 0.002 | 50 | 0.7 |
| N-palmitoylglycine | 314.2 | 0.002 | 50 | 0.7 |

### Example 8

### Production of Fatty Acid Amino Acid Adducts by E. coli Strains with Deletion in the fadE Gene, which Strains Overexpress the synUcTE and fadD Genes in Combination with hGLYAT2 or hGLYAT3 in a Parallel Fermentation System

The strains generated in Example 4 were used for studying their ability to produce fatty acid amino acid adducts from glucose. For this purpose, the strain was cultured both in a shake flask and in a fed-batch fermentation. The fermentation was carried out in a parallel fermentation system from DASGIP with 8 bioreactors.

The production cells were prepared as described in Example 6.

The fermentation was performed using 1 I reactors equipped with overhead stirrers and impeller blades. pH and pO₂ were measured online for process monitoring. OTR/CTR measurements served for estimating the metabolic activity and cell fitness, inter alia.

The pH electrodes were calibrated by means of a two-point calibration using standard solutions of pH 4.0 and pH 7.0, as specified in DASGIP's technical instructions. The reactors were provided with the necessary sensors and connections as specified in the technical instructions, and the agitator shaft was fitted. The reactors were then charged with 300 mL of water and autoclaved for 20 min at 121° C. to ensure sterility. The pO₂ electrodes were connected to the measuring amplifiers and polarized overnight (for at least 6 h). Thereafter, the water was removed under a clean bench and replaced by M9 medium (pH 7.4) composed of KH₂PO₄ 3.0 g/l, Na₂HPO₄ 6.79 g/l, NaCl 0.5 g/l, NH₄Cl 2.0 g/l, 2 mL of a sterile 1 M MgSO₄*7H₂O solution and 1 mL/l of a filter-sterilized trace element stock solution (composed of HCl (37%) 36.50 g/L, MnCl₂*4H₂O 1.91 g/L, ZnSO₄*7H₂O 1.87 g/L, ethylenediaminetetraacetic acid dihydrate 0.84 g/L, H₃BO₃ 0.30 g/L, Na₂MoO₄*2H₂O 0.25 g/L, CaCl₂*2H₂O 4.70 g/L, FeSO₄*7H₂O 17.80 g/L, CuCl₂*H₂O 0.15 g/L) with 15 g/L glucose as the carbon source (added by metering in 30 mL/L of a sterile feed solution composed of 500 g/L glucose, 1.3% (w/v) MgSO₄*7H₂O) supplemented with 100 mg/L spectinomycin and 3 mL/l DOW1500.

Thereafter, the pO₂ electrodes were calibrated to 100% with a one-point calibration (stirrer: 400 rpm/aeration: 10 sl/h air), and the feed, correction agent and induction agent lines were cleaned by “cleaning in place” as specified in the technical instructions. To this end, the tubes were rinsed first with 70% ethanol, then with 1 M NaOH, then with sterile fully-demineralized water and, finally, filled with the respective media.

Using the E. coli strain of Example 4, a dilution streak was first performed with a cryoculture on an LB agar plate supplemented with 100 mg/l spectinomycin, and the plate was incubated for approximately 16 h at 37° C. LB medium (10 mL in a 100-mL baffle flask) supplemented with 100 mg/l spectinomycin was then inoculated with a single colony and the culture was grown overnight at 37° C. and 200 rpm for approximately 16 h. Thereafter, this culture was used for a second preculture stage with an initial OD of 0.2 in 50 mL of M9 medium, composed of KH₂PO₄ 3.0 g/l, Na₂HPO₄ 6.79 g/l, NaCl 0.5 g/l, NH₄Cl 2.0 g/l, 2 mL of a sterile 1 M MgSO₄*7H₂O solution and 1 mL/l of a filter-sterilized trace element stock solution (composed of HCl (37%) 36.50 g/L, MnCl₂*4H₂O 1.91 g/L, ZnSO₄*7H₂O 1.87 g/L, ethylenediaminetetraacetic acid dihydrate 0.84 g/l, H₃BO₃ 0.30 g/l, Na₂MoO₄*2H₂O 0.25 g/L, CaCl₂*2H₂O 4.70 g/L, FeSO₄*7H₂O 17.80 g/L, CuCl₂*2H₂O 0.15 g/L) supplemented with 20 g/L glucose as carbon source (added by metering in 40 mL/L of a sterile feed solution composed of 500 g/L glucose) together with the above-described antibiotics was transferred into a 500-mL baffle flask and incubated for 8-12 h at 37° C./200 rpm.

To inoculate the reactors with an optical density of 0.1, the OD₆₀₀ of the second preculture stage was measured and the amount of culture required for the inoculation was calculated. The required amount of culture was placed into the heated and aerated reactor with the aid of a 5-mL syringe through a septum.

The standard program shown in Table 6a-c was used:

TABLE 6
Standard program for use of heated and aerated reactor in Example 8
a)

| DO controller | | pH controller | | |
|---|---|---|---|---|
| Preset | 0% | Preset | 0 | mL/h |
| P | 0.1 | P | 5 | |
| Ti | 300 s | Ti | 200 | s |
| Min | 0% | Min | 0 | mL/h |
| Max | 100% | Max | 40 | mL/h |

b)

| | | | XO2 | | | F | | |
|---|---|---|---|---|---|---|---|---|
| | | | (gas | | | (gas | | |
| N (Rotation) | from | to | mixture) | from | to | flow) | from | to |
| Growth | 0% | 30% | Growth | 0% | 100% | Growth | 15% | 80% |
| and | 400 rpm | 1500 rpm | and | 21% | 21% | and | 6 sl/h | 72 sl/h |
| biotrans- | | | biotrans- | | | biotrans- | | |
| formation | | | formation | | | formation | | |

c)

| Script | |
|---|---|
| Trigger fires | 31% DO (1/60 h) |
| Induction IPTG | 2 h after the feed start |
| Feed trigger | 50% DO |
| Feed rate | 3 [mL/h] |

The pH was adjusted unilaterally to pH 7.0 with 12.5% strength ammonia solution. During the growth phase and the biotransformation, the dissolved oxygen (pO₂ or DO) in the culture was adjusted to at least 30% via the stirrer speed and the aeration rate. After the inoculation, the DO dropped from 100% to these 30%, where it was maintained stably for the remainder of the fermentation.

The fermentation was carried out as a fed batch, the feed start as the beginning of the feed phase with 5 g/l*h glucose feed, composed of 500 g/l glucose, 1.3% (w/v) MgSO₄*7H₂O, being triggered via the DO peak which indicates the end of the batch phase. From the feed start onwards, the temperature was reduced from 37° C. to 30° C. 2 h after the feed start, the expression was induced with 1 mM IPTG.

To quantify lauroyl, myristoyl and palmitoyl glycinate, samples were taken 47 h and 64 h after the start of the fermentation. These samples were prepared for analysis, and analyzed as described in Example 7.

It has been possible to demonstrate that strain E. coli W3110 ΔfadE pJ294{Ptac}[synUcTE]/pCDF{Ptac}[hGLYAT2(co_Ec)_fadD_Ec] is capable of forming lauroyl glycinate from glucose. The results are shown in Tables 7 and 8.

TABLE 7
Quantification of fatty acid glycinates
after 47 and 64 h fermentation time.

| | Ion [M + H] | Scan range | Scan time | Resolution |
|---|---|---|---|---|
| Analyte | [m/z] | [m/z] | [ms] | Q3 |
| N-Lauroylglycine | 258.2 | 0.002 | 50 | 0.7 |
| N-Myristoylglycine | 286.2 | 0.002 | 50 | 0.7 |
| N-Palmitoylglycine | 314.2 | 0.002 | 50 | 0.7 |

TABLE 8
Production of fatty acids after 47 and 64 hours' fermentation time.

| Fermentation | C_{Lauroyl glycinate} | C_{Myristoyl glycinate} | C_{Palmitoyl glycinate} | C_{Glycine} | C_{Octanoic acid} | C_{Decanoic acid} |
|---|---|---|---|---|---|---|
| time [h] | [g/L] | [g/L] | [g/L] | [g/L] | [g/L] | [g/L] |
| 47 | 0.63 | 0.07 | n.d. | n.d. | n.d. | 0.001 |
| 64 | 1.06 | 0.11 | n.d. | n.d. | n.d. | n.d. |

| Fermentation | C_{Lauric acid} | C_{Myristic acid} | C_{Palmitoleic acid} | C_{Palmitic acid} | C_{Oleic acid} | C_{Stearic acid} |
|---|---|---|---|---|---|---|
| time [h] | [g/L] | [g/L] | [g/L] | [g/L] | [g/L] | [g/L] |
| 47 | n.d. | n.d. | n.d. | 0.002 | 0.006 | 0.001 |
| 64 | 0.001 | n.d. | n.d. | n.d. | 0.002 | n.d. |

n.d.: not determined

### Example 9

The strain of Example 5 was fermented in a fed-batch fermentation to study the ability of linking lauric acid and glycine to give lauroyl glycinate. This fermentation was carried out in a parallel fermentation system from DASGIP with 8 bioreactors.

The experimental setting was as described in Example 8 except that 100 g/l glycine in demineralized water and 100 g/l laurate in lauric acid methyl ester were fed rather than glucose. To quantify lauroyl, myristoyl and palmitoyl glycinate in fermentation samples, samples were taken 23 h and 42 h after the start of the fermentation. These samples were prepared for analysis, and analyzed as described in Example 7. The results are shown in Tables 9 and 10.

It has been possible to demonstrate that the strain E. coli W3110 ΔfadE pCDF{Ptac}[hGLYAT2(co_Ec)_fadD_Ec] {Plavuv5} [alkLmod1] is capable of linking lauric acid and glycine and of producing lauroyl glycinate.

TABLE 9
Production of lauroyl glycinate after fermentation for 23
and 42 hours with feeding of lauric acid and glycine.

| Fermentation | C_{Lauroyl glycinate} | C_{Myristoyl glycinate} | C_{Palmitoyl glycinate} | C_{Glycine} | C_{Octanoic acid} | C_{Decanoic acid} |
|---|---|---|---|---|---|---|
| time [h] | [g/L] | [g/L] | [g/L] | [g/L] | [g/L] | [g/L] |
| 21 | 1.02 | n.d. | n.d. | n.d. | n.d. | n.d. |
| 40 | 1.78 | n.d. | n.d. | n.d. | n.d. | n.d. |

| Fermentation | C_{Lauric acid} | C_{Myristic acid} | C_{Palmitoleic acid} | C_{Palmitic acid} | C_{Oleic acid} | C_{Stearic acid} |
|---|---|---|---|---|---|---|
| time [h] | [g/L] | [g/L] | [g/L] | [g/L] | [g/L] | [g/L] |
| 21 | 6.38 | n.d. | n.d. | n.d. | n.d. | n.d. |
| 40 | 6.79 | n.d. | n.d. | n.d. | n.d. | n.d. |

TABLE 10
Production of lauroyl glycinate after fermentation for 23 and 42 hours
without feeding of lauric acid and glycine (negative control).

| Fermentation | C_{Lauryl glycinate} | C_{Myristyl glycinate} | C_{Palmityl glycinate} | C_{Glycine} | C_{Octanoic acid} | C_{Decanoic acid} | C_{Lauric acid} |
|---|---|---|---|---|---|---|---|
| time [h] | [g/L] | [g/L] | [g/L] | [g/L] | [g/L] | [g/L] | [g/L] |
| 21 | n.d. | n.d. | n.d. | n.d. | n.d. | n.d. | n.d. |
| 40 | n.d. | n.d. | n.d. | n.d. | n.d. | n.d. | n.d. |

| Fermentation | C_{Myristic acid} | C_{Palmitoleic acid} | C_{Palmitic acid} | C_{Oleic acid} | C_{Stearic acid} |
|---|---|---|---|---|---|
| time [h] | [g/L] | [g/L] | [g/L] | [g/L] | [g/L] |
| 21 | n.d. | n.d. | n.d. | n.d. | n.d. |
| 40 | n.d. | n.d. | n.d. | n.d. | n.d. |

### Example 10

### Generation of Vectors for Expression of the Homo sapiens Genes hGLYAT3 and hGLYAT2 in E. coli Strains Producing Fatty Acids Via Malonyl-CoA and Acetyl-CoA

To generate vectors for the expression of the Homo sapiens genes hGLYAT2 (SEQ ID NO: 4) or hGYLAT3 (SEQ ID NO: 5) in the fatty acid producing strains listed in Table 13 below. (From Table 3.2 of W02014026162Δ1), the genes hGLYAT2 and hGYLAT3 were first amplified. The gene hGLYAT2 was amplified from the plasmid pCDF{Ptac}[hGLYAT2(co_Ec)-fadD_Ec]{Placuv5}[alkLmod1] (SEQ ID NO: 13) and the hGYLAT3 gene was amplified from the plasmid pCDF{Ptac}[hGLYAT3(co_Ec)-fadD_Ec] (SEQ ID No: 9) by means of sequence-specific oligonucleotides. The PCR products were cleaved with the restriction endonucleases NotI and SacI and ligated in the correspondingly cleaved vector pET-28b (SEQ ID NO: 14). The correct insertion of the target genes was checked by restriction analysis and the authenticity of the introduced genes was verified by DNA sequencing. The resulting expression vectors were named pET-28b{Ptac}[hGLYAT2(co_Ec)] (SEQ ID No: 15) and pET-28b{Ptac}[hGLYAT3(co_Ec)] (SEQ ID No: 16).

### Example 11

### Production of Fatty Acid Amino Acid Adducts Via Malonyl-CoA and Acetyl-CoA by Strains Overexpressing hGLYAT2 or hGLYAT3 in a Shake Flask Experiment

The vectors produced according to Example 10 were then used to generate a microorganism strain from Table 11 below (OPX Biotechnologies Inc., USA) using any transformation method known in the art. In particular, the methods provided in section IV of W02014026162Δ1 were used.

The strains generated were used for studying their ability to produce fatty acids, in particular amino acid adducts from glucose. For this purpose, the strains were transformed with the vectors pET-28b{Ptac}[hGLYAT2(co_Ec)] (SEQ ID NO: 15) and pET-28b{Ptac}[hGLYAT3(co_Ec)] (SEQ ID NO: 16) and cultured in shake flasks (Subsection C of section IV of W02014026162Δ1). Strain BXF_031 (OPX Biotechnologies Inc., USA) harbouring the empty vector pET-28b was used as a control.

Triplicate evaluations were performed. Briefly, overnight starter cultures were made in 50 mL of Terrific Broth including the appropriate antibiotics and incubated 16-24 hours at 30° C., while shaking at 225 rpm. These cultures were used to inoculate 150 mL cultures of each strain in SM11 minimal medium to an OD₆₀₀ of 0.8 and 5% TB culture carryover as starting inoculum, and antibiotics. 1 L SM11 medium consists of: 2 mL FM10 Trace Mineral Stock, 2.26 mL 1M MgSO₄, 30 g glucose, 200 mM MOPS (pH 7.4), 1 g/L yeast extract, 1.25 mL VM1 Vitamin Mix, 0.329 g K₂HPO₄, 0.173 g KH₂PO₄, 3 g (NH₄)₂SO₄, 0.15 g citric acid (anhydrous); FM10 Trace Mineral Stock consists of: 1 mL of concentrated HCl, 4.9 g CaCl₂*2H₂O, 0.97 g FeCl₃*6H₂O, 0.04 g CoCl₂*6H₂O, 0.27 g CuCl₂*2H₂O, 0.02 g ZnCl₂, 0.024 g Na₂MoO₄*2H₂O, 0.007 g H₃BO₃, 0.036 g MnCl2*4H₂O, Q.S. with DI water to 100 mL; VM1 Vitamin Mix Solution consists of: 5 g Thiamine, 5.4 g Pantothenic acid, 6.0 g Niacin, 0.06 g, Q.S. with DI water to 1000 mL. All ingredients for the culture mediums used in this example are provided in (Subsection A of section IV of W02014026162A1).

Cultures were incubated for 2 hours at 30° C., while shaking at 225 rpm. After 2 hours, the cells were washed with SM11 (SM11 medium without phosphate). Cells were twice spun down (4,000 rpm, 15 min), the supernatant decanted, the pellet re-suspended in 150 mL of SM11 (SM11 medium without phosphate). The cultures were used to inoculate 3×50 mL of each strain in SM11 (no phosphate). The cultures were grown at 30° C. for approximately 2 h to an OD₆₀₀ of 1.0-1.5 after 2 h cells and shifted to 37° C. and samples removed periodically for product measurement over the course of 72 hrs.

The quantification of N-lauroylglycine, N-myristoylglycine and N-palmitoylglycine and the detection of other acylamino acids in fermentation samples was performed by HPLC-ESI/MS as described in Example 7.

TABLE 11
List of microorganism strains that were used to introduce the genes hGLYAT2 or hGLYAT3
in examples 10 and 11. The method of production and the sequences of the strains
are provided in Table 3.2 of WO2014026162A1 (OPX Biotechnologies Inc., USA).

| Strain | | | SEQ ID |
|---|---|---|---|
| designation | Host | Plasmid | NOs. |
| BXF_0012 | BX_864 | 1)pBMT-3_ccdAB | 17 |
| BXF_0013 | BX_864 | 1)pBMT-3_ccdAB_P_{T7}-′tesA | 18 |
| BXF_0014 | BX_864 | 1)pBMT-3_ccdAB_P_{T7}-nphT7-hbd-crt-ter | 19 |
| BXF_0015 | BX_864 | 1)pBMT-3_ccdAB_P_{T7}-′tesA_PT7-nph_{T7}-hbd-crt-ter | 20 |
| BXF_0020 | BX_860 | 1)pBMT-3_ccdAB_PT7-′tesA_PT7-nphT7-hbd-crt-ter | 20 |
| BXF_0021 | BX_876 | 1)pBMT-3_ccdAB_PT7-′tesA_PT7-nphT7-hbd-crt-ter | 20 |
| BXF_0022 | BX_874 | 1)pBMT-3_ccdAB | 17 |
| BXF_0023 | BX_874 | 1)pBMT-3_ccdAB_PT7-′tesA | 18 |
| BXF_0024 | BX_874 | 1)pBMT-3_ccdAB_PT7-′tesA_PT7-nphT7-hbd-crt-ter | 20 |
| BXF_0025 | BX_875 | 1)pBMT-3_ccdAB | 17 |
| BXF_0026 | BX_875 | 1)pBMT-3_ccdAB_PT7-′tesA | 18 |
| BXF_0027 | BX_875 | 1)pBMT-3_ccdAB_PT7-′tesA_PT7-nphT7-hbd-crt-ter | 20 |
| BXF_0028 | BX_878 | 1)pBMT-3ccdAB-T7-′tesA-PT7_nphT7_hbd_crt_ter | 20 |
| BXF_0028 | BX_878 | 1)pBMT-3_ccdAB_PT7-′tesA_PT7-nphT7-hbd-crt-ter | 20 |
| BXF_0029 | BX_879 | 1)pBMT-3_ccdAB_PT7-′tesA_PT7-nphT7-hbd-crt-ter | 20 |
| BXF_0030 | BX_881 | 1)pBMT-3_ccdAB_PT7-′tesA_PT7-nphT7-hbd-crt-ter | 20 |
| BXF_0031 | BX_864 | 1)pBMT-3_ccdAB_PT7-′tesA_PT7-nphT7-hbd-crt-ter | 20 |
| | | 2)pET-28b(empty vector) | 21 |
| BXF_0033 | BX_878 | 1)pBMT-3_ccdAB_PT7-nphT7-hbd-crt-ter | 19 |
| BXF_0034 | BX_879 | 2)pBMT-3_ccdAB_PT7-nphT7-hbd-crt-ter | 19 |

### Example 12

### Generation of a Vector for Deletion of the gcvTHP Operon in Escherichia coli W3110 ΔfadE

To generate a vector for the deletion of the gcvTHP operon of E. coli W3110, which encodes a glycine cleavage system (gcvT: aminomethyltransferase, tetrahydrofolate-dependent, subunit (T protein) of glycine cleavage complex; gcvH: glycine cleavage complex lipoylprotein; gcvP: glycine decarboxylase, PLP-dependent, subunit (protein P) of glycine cleavage complex), approx. 500 bp upstream and downstream of the gcvTHP operon were amplified via PCR. The upstream region of gcvTHP was amplified using the oligonucleotides o-MO-40 (SEQ ID No:22). And o-MO-41 (SEQ ID No:23) The downstream region of gcvTHP was amplified using the oligonucleotides o-MO-42 (SEQ ID No:24) and o-MO-43 (SEQ ID No:25). The PCR procedure is described above in Example 3.

In each case PCR fragments of the expected size could be amplified (PCR 1,553 bp, (SEQ ID No:26); PCR 2,547 bp, SEQ ID No:27). The PCR samples were separated via agarose gel electrophoresis and DNA fragments were isolated with QiaQuick Gel extraction Kit (Qiagen, Hilden). The purified PCR fragments were cloned into the vector pKO3 (SEQ ID No:28), and cut with BamHI using the Geneart® Seamless Cloning and Assembly Kit (Life Technologies, Carlsbad, Calif., USA). The assembled product was transformed into chemically competent E. coli DH5a cells (New England Biolabs, Frankfurt). Procedure of PCR purification, in-vitro cloning and transformation were carried out according to manufacturer's manual. The correct insertion of the target genes was checked by restriction analysis and the authenticity of the introduced DNA fragments was verified by DNA sequencing. The resulting knock-out vector was named pKO3 delta gcvTHP (SEQ ID No:29).

The construction of strain E. coli W3110 ΔfadE ΔgcvTHP was carried out with the help of pKO3 delta gcvTHP using the method described in Link et al., 1997. The DNA sequence after deletion of gcvTHP is SEQ ID No: 30. The E. coli strain W3110 ΔfadE ΔgcvTHP was transformed with the plasmid pCDF{Ptac}[hGLYAT2(co_Ec)-fadD_Ec]{Placuv5}[alkLmod1] (SEQ ID No: 13, Example 3) by means of electroporation and plated onto LB-agar plates supplemented with spectinomycin (100 μg/mL). Transformants were checked for the presence of the correct plasmids by plasmid preparation and analytic restriction analysis. The resulting strain was named E. coli W3110 ΔfadE ΔgcvTHP pCDF{Ptac}[hGLYAT2(co_Ec)-fadD_Ec]{Placuv5}[alkLmod1].

### Example 13

### Production of Lauroylglycinate by E. coli Strains with Deletion in the fadE or fadE I gcvTHP Gene, Overexpressing the hGLYAT2, fadD and alkL Genes

The strain generated in Example 1 was used to study its ability to produce more lauroylglycinate, in comparison to the reference strain without gcvTHP deletion.

Starting from a −80° C. glycerol culture, the strains to be studied were first plated onto an LB-agar plate supplemented with 100 μg/mL spectinomycin and incubated overnight at 37° C. Starting from a single colony in each case, the strains were then grown as a 5-mL preculture in LB-broth, Miller (Merck, Darmstadt) supplemented with 100 μg/mL spectinomycin. The further culture steps were performed in M9-FIT medium. The medium, composed of 38 mM disodium hydrogenphosphate dihydrate, 22 mM potassium dihydrogenphosphate, 8.6 mM sodium chloride, 37 mM ammonium chloride, 2 mM magnesium sulphate heptahydrate (all chemicals from Merck, Darmstadt), 5% (w/v) maltodextrin solution (dextrose equivalent 13.0-17.0, Sigma Aldrich, Taufkirchen), 1% (w/v) amyloglycosidase from Aspergillus niger (Sigma-Aldrich, Taufkirchen), 1 drop Delamex 180 (Bussetti & Co, Wien) and 0.1% (v/v) trace element solution, was brought to pH 7.4 with 25% strength ammonium hydroxide solution. The trace element solution added, composed of 9.7 mM manganese(II) chloride tetrahydrate, 6.5 mM zinc sulphate heptahydrate, 2.5 mM sodium-EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate, dissolved in 1 M hydrochloric acid (all chemicals from Merck, Darmstadt) was filter-sterilized before being added to the M9 medium. 20 mL of M9 medium supplemented with 100 μg/mL spectinomycin were introduced into baffled 100-mL Erlenmeyer flasks and inoculated with 0.5 mL preculture. The flasks were cultured at 37° C. and 200 rpm in a shaker-incubator. After a culture time of 8 hours, 50 mL of M9 medium supplemented with 100 μg/mL spectinomycin and were introduced into a baffled 250-mL Erlenmeyer flask and inoculated with the 10-mL culture to achieve an optical density (600 nm) of 0.1. The flasks were cultured at 37° C. and 200 rpm in a shaker-incubator. When an optical density (600 nm) of 0.6 to 0.8 was reached, gene expression was induced by addition of 1 mM IPTG. The strains were cultured for a further 48 hours at 37° C. and 200 rpm. 1-3 h after the induction, 6 g/L glycine and 6 g/L lauric acid (dissolved in lauric acid methyl ester) were added to the cultures. After 0 h and 24 h cultivation samples were taken, and lauroylglycinate, lauric acid and glycine present were analysed. The results are shown in FIGS. 4 and 5. It has been possible to demonstrate that both E. coli strains W3110 ΔfadE pCDF{Ptac}[hGLYAT2(co_Ec)-fadD_Ec]{Placuv5}[alkLmod1] and E. coli strain W3110 ΔfadE ΔgcvTHP pCDF{Ptac}[hGLYAT2(co_Ec)-fadD_Ec]{Placuv5}[alkLmod1] are capable of forming lauroylglycinate. But in the new strain background ΔfadE ΔgcvTHP higher amounts of lauroylglycinate were synthesized and higher amounts of glycine are detected. It appears that less glycine is metabolized in the ΔgcvTHP background and can be used for lauroylglycinate synthesis.

### Example 14

### Generation of a Vector for Deletion of the glyA Gene in Escherichia coli W3110 ΔfadE

To generate a vector for the deletion of the glyA-gene encoding a component of the Glycine hydroxymethyltransferase of E. coli W3110 approx. 500 bp upstream and downstream of the glyA-gene were amplified via PCR. The upstream region of glyA was amplified using the oligonucleotides o-MO-44 (SEQ ID No:31) and o-MO-45 (SEQ ID No:32). The downstream region of glyA was amplified using the oligonucleotides o-MO-46 (SEQ ID No:33) and o-MO-47 (SEQ ID No:34).

In each case PCR fragments of the expected size could be amplified (PCR 1,546 bp, (SEQ ID No:35); PCR 2,520 bp, SEQ ID No:36). The PCR samples were separated via agarose gel electrophoresis and DNA fragments were isolated with QiaQuick Gel extraction Kit (Qiagen, Hilden). The purified PCR fragments were assembled via a crossover PCR. The generated fragment was purified and subcloned into the cloning vector pCR®-Blunt IITOPO (Life technologies) according to manufacturer's manual. To clone the fragment into the target plasmid pKO3 (SEQ ID No:28) it was amplified with flanking BamHI restriction sites, using the oligonucleotides o-MO-52 (SEQ ID No:37) and o-MO-53 (SEQ ID No:38). The purified, BamHI cleaved PCR 3 fragment (SEQ ID No:39) was ligated into the correspondingly cleaved vector pKO3 (SEQ ID No:28). The assembled product was transformed into chemically competent E. coli DH5α cells (New England Biolabs, Frankfurt). Procedure of PCR purification, in-vitro cloning and transformation were carried out according to manufacturer's manual. The correct insertion of the target genes was checked by restriction analysis and the authenticity of the introduced genes was verified by DNA sequencing. The resulting knock-out vector was named pKO3 delta glyA (SEQ ID No:40).

The construction of strain E. coli W3110 ΔfadE ΔglyA was carried out with the help of pKO3 delta glyA using the method described in Link et al., 1997. SEQ ID No:41 is the DNA sequence after deletion of glyA. The E. coli strain W3110 ΔfadE ΔgcvTHP was transformed with the plasmid pCDF{Ptac}[hGLYAT2(co_Ec)-fadD_Ec]{Placuv5}[alkLmod1] (SEQ ID No: 13 from Example 3), by means of electroporation and plated onto LB-agar plates supplemented with spectinomycin (100 μg/mL). Transformants were checked for the presence of the correct plasmids by plasmid preparation and analytic restriction analysis. The resulting strain was named E. coli W3110 ΔfadE ΔglyA pCDF{Ptac}[hGLYAT2(co_Ec)-fadD_Ec]{Placuv5}[alkLmod1].

### Example 15

### Generation of a Vector for Deletion of the ItaE Gene in Escherichia coli W3110 ΔfadE

To generate a vector for the deletion of the ItaE-gene encoding the L-allo-threonine aldolase of E. coli W3110 approximately 500 bp upstream and downstream of the ItaE were amplified via PCR as described above. The upstream region of ItaE was amplified using the oligonucleotides ItaE-UP_fw (SEQ ID No:42) and ItAE-UP-XhoI_rev (SEQ ID No:43). The downstream region of ItaE was amplified using the oligonucleotides ItaE-DOWN_fw (SEQ ID No:44) and ItaE-DOWN_rev (SEQ ID No:45).

In each case PCR fragments of the expected size could be amplified (PCR 4,550 bp, (SEQ ID No:46); PCR 5, 536 bp, SEQ ID No:47). The PCR samples were separated via agarose gel electrophoresis and DNA fragments were isolated with QiaQuick Gel extraction Kit (Qiagen, Hilden). The purified PCR fragments were assembled via a crossover PCR. The generated fragment was purified and cloned into the cloning vector pCR®-Blunt IITOPO (Life technologies) according to manufacturer's manual. To clone the fragment into the target plasmid pKO3 (SEQ ID No:28) it was amplified with flanking BamHI restriction sites, using the oligonucleotides o-MO-54 (SEQ ID No:48) and o-MO-55 (SEQ ID No:49). The purified, BamHI cleaved PCR 6 fragment (SEQ ID No:50) was ligated into the correspondingly cleaved vector pKO3 (SEQ ID No:28). The assembled product was transformed into chemically competent E. coli DH5a cells (New England Biolabs, Frankfurt). Procedure of PCR purification, in-vitro cloning and transformation were carried out according to manufacturer's manual. The correct insertion of the target genes was checked by restriction analysis and the authenticity of the introduced genes was verified by DNA sequencing. The resulting knock-out vector was named pKO3 delta ItaE (SEQ ID No:51).

The construction of strain E. coli W3110 ΔfadE ΔltaE was carried out with the help of pKO3 delta ItaE using the method described in Link et al., 1997 (Link A J, Phillips D, Church G M. J. Bacteriol. 1997. 179(20). The DNA sequence after deletion of ItaE is described in SEQ ID No:52). The E. coli strain W3110 ΔfadE ΔltaE was transformed with the plasmid pCDF{Ptac}[hGLYAT2(co_Ec)-fadD_Ec]{Placuv5}[alkLmod1] (SEQ ID NO: 13 from Example 3), by means of electroporation and plated onto LB-agar plates supplemented with spectinomycin (100 μg/mL). Transformants were checked for the presence of the correct plasmids by plasmid preparation and analytic restriction analysis. The resulting strain was named E. coli W3110 ΔfadE ΔltaE pCDF{Ptac}[hGLYAT2(co_Ec)-fadD_Ec]{Placuv5}[alkLmod1].

### Example 16

### LC-ESI/MS²-based quantification of lauric acid

Quantification of lauric acid in fermentation samples was carried out by means of LC-ESI/MS² on the basis of an external calibration for lauric acid (0.1-50 mg/L) and by using the internal standard d3-LS.

The following instruments were used:
HPLC system 1260 (Agilent; Boblingen) with Autosampler (G1367E), binary pump (G1312B) and thermo-statted column (G1316A)
Mass spectrometer TripelQuad 6410 (Agilent; Boblingen) with ESI source
HPLC column: Kinetex C18, 100×2.1 mm, particle size: 2.6 μm, pore size 100 Å (Phenomenex; Aschaffenburg)
Pre-column: KrudKatcher Ultra HPLC In-Line Filter; 0.5 μm filter depth and 0.004 mm inner diameter (Phenomenex; Aschaffenburg)

The samples were prepared by pipetting 1900 μL of solvent (80% (v/v) ACN, 20% double-distilled H₂O (v/v), +0.1% formic acid) and 100 μL of sample into a 2 mL reaction vessel. The mixture was vortexed for approx. 10 seconds and then centrifuged at approx. 13 000 rpm for 5 min. The clear supernatant was removed using a pipette and analysed after appropriate dilution with a diluent (80% (v/v) ACN, 20% double-distilled H₂O (v/v), +0.1% formic acid). In each case, 100 μL of ISTD were added to 900 μL of sample (10 μL with a sample volume of 90 μL).

HPLC separation was carried out using the above-mentioned column and pre-column. The injection volume is 1.0 μL, the column temperature 50° C., the flow rate is 0.6 mL/min. the mobile phase consists of eluent A (0.1% strength (v/v) aqueous formic acid) and eluent B (acetonitrile with 0.1% (v/v) formic acid). The gradient shown it Table 14 was utilized:

TABLE 12
Concentrations of Eluent A and B used in Example 16

| Time | Eluent A | Eluent B |
|---|---|---|
| [min] | [%] | [%] |
| 0 | 85 | 15 |
| 1 | 85 | 15 |
| 5 | 2 | 98 |
| 8 | 2 | 98 |
| 8.1 | 85 | 15 |
| 12 | 85 | 15 |

ESI-MS² analysis was carried out in positive mode with the following parameters of the ESI source:
Gas temperature 320° C.
Gas flow 11 L/min
Nebulizer pressure 50 psi
Capillary voltage 4000 V

Detection and quantification of lauric acid was carried out with the following MRM parameters.

TABLE 13
MRM parameters used in detection and quantification of lauric acid

| | Precursor ion | Product ion | Collision energy |
|---|---|---|---|
| Analyte | [m/z] | [m/z] | [eV] |
| LS | 201.1 | 201.1 | 110 |
| d3-LS | 204.1 | 204.1 | 110 |

### Example 17

### Detection of Glycine

Detection of glycine was performed via derivatization with ortho-phthaldialdehyde (OPA) and UV/VIS detection using an Agilent 1200 HPLC system.

200 μL of a homogeneous fermentation broth simple was mixed with 1800 μL of 30% (v/v) 1-propanol, vortexed for 10 s and subsequently centrifuged at 13,000×g for 5 min. The supernatant was removed and used for HPLC analysis using the following parameters:

| Mobile phase: | Eluent A | | | |
|---|---|---|---|---|
| | 2.5 mL acetic acid per 1 L distilled water, pH | | | |
| | adjustment with NaOH @ 6.0 | | | |
| | Eluent B | | | |
| | Methanol | | | |
| Column: | Luna 5μ C8 100 A (100 × 4.6 mm); | | | |
| | Phenomenex | | | |
| Column oven | 40° C. | | | |
| temperature: | | | | |
| Flow: | 1.0 mL/min | | | |
| Gradient: | Time | % B | Flow | Max. Press. |
| | 0.0 | 30.0 | 1.0 | 400 |
| | 1.0 | 30.0 | 1.0 | 400 |
| | 17.0 | 90.0 | 1.0 | 400 |
| | 19.5 | 90.0 | 1.0 | 400 |
| | 19.6 | 30.0 | 1.0 | 400 |
| | 20.5 | 30.0 | 1.0 | 400 |
| Run time: | 22 min | | | |
| Detector: | DAD | | | |
| | 334 nm | | | |
| | Spectrum | | | |
| | Store: all | | | |
| | Range: 200-400 nm | | | |
| | step 2 nm | | | |
| | FLD (excitation @ 330 nm; emission @ 450 nm, | | | |
| | PMT gain 13) | | | |
| Derivatization: | automatically with injection program: | | | |
| | Inject Programm | | | |
| | # | Command | | |

| | |
|---|---|
| 1 | DRAW 4.5 μL from Vial 1*, def. |
| | speed, def. offset |
| 2 | DRAW 1.5 μL from sample, def. |
| | speed, def. offset |
| 3 | DRAW 0.5 μL from air, def. speed |
| 4 | NEEDLE wash in flush Port. 15.0 sec |
| 5 | DRAW 4.5 μL from Vial 1, def. |
| | speed, def. offset |
| 6 | MIX 11.0 μL in seat, def. |
| | speed, 1 times |
| 7 | WAIT 1.00 min |
| 8 | INJECT |
| 9 | WAIT 0.50 min |
| 10 | VALVE bypass |
| 11 | Draw 100.0 μL from Vial 2*, |
| | def. speed, def. offset |
| 12 | Eject 100.0 μL from Vial 2, |
| | def. speed, def. offset |
| 13 | Valve mainpass |

*vial 1 contains OPA reagent (see below); vial 2 contains water

### Preparation of OPA Reagent

100 mg o-phthaldialdehyde was dissolved in 1 mL methanol and subsequently 0.4 mM borate buffer (pH 10.4) was added to give 10 mL. Subsequently, 50 μL mercaptoethanol was added and the reagent stored at 4° C. Additional 10 μL mercaptoethanol was added before use.

Preparation of Borate Buffer (0.4 mM H₃BO₄):

38.1 g Na₂B₄O₇*10H₂O (0.1 mol) were dissolved in 1 L distilled water and the pH adjusted to 10.4 M NaOH auf 10.4 eingestellt. Subsequently, 1 mL 25% Brij35 (v/v) was added.

Retention time: Glycine: 7.153 min

### Example 18

### Generation of Vectors for the Expression of hGLYAT2-Homologs

To generate vectors for the expression of N-acyltransferases of different organisms, variants found in the NCBI databases with homology to HGLYAT2 were synthesized and codon-optimized for E. co/i. These were glycine N-acyltransferase-like protein 2 isoform 1 of Nomascus leucogenys (NI, XP_003275392.1, SEQ ID No:53), glycine N-acyltransferase-like protein 2 of Saimiri boliviensis (Sb, XP_003920208.1, SEQ ID No:54), glycine-N-acyltransferase-like 2 of Felis catus (Fc, XP_003993512.1, SEQ ID No:55), glycine N-acyltransferase-like protein 2 of Bos taurus (Bt, NP_001178259.1, SEQ ID No:56), and glycine N-acyltransferase of Mus musculus (Mm, NP_666047.1, SEQ ID No:57).

The hGLYAT2-gene of pCDF{Ptac}[hGLYAT2(co_Ec)-fadD_Ec]{Placuv5}[alkLmod1] (SEQ ID No: 13 of Example 3) was replaced by this variants as follows: The synthesized DNA fragments were digested with the restriction endonucleases BamHI and AsiSI and ligated into the correspondingly cut pCDF{Ptac}[hGLYAT2(co_Ec)-fadD_Ec]{Placuv5}[alkLmod1].

The correct insertion of the target genes was checked by restriction analysis and the authenticity of the introduced genes was verified by DNA sequencing. The resulting expression vectors were named:

pCDF{Ptac}[GLYAT_NI(co_Ec)-fadD_Ec]{Placuv5}[alkLmod1]
pCDF{Ptac}[GLYAT_Sb(co_Ec)-fadD_Ec]{Placuv5}[alkLmod1]
pCDF{Ptac}[GLYAT_Fc(co_Ec)-fadD_Ec]{Placuv5}[alkLmod1]
pCDF{Ptac}[GLYAT_Bt(co_Ec)-fadD_Ec]{Placuv5}[alkLmod1]
pCDF{Ptac}[GLYAT_Mm(co_Ec)-fadD_Ec]{Placuv5}[alkLmod1]

### Example 19

### Production of Lauroylglycinate by E. coli Strains with Deletion in the fadE Gene, Overexpressing the hGLYAT-Variants, fadD and alkL Genes

The strains generated in Example 18 were used to study their ability to produce lauroylglycinate, in comparison to the reference strain expressing hGLYAT2 harbouring the plasmid pCDF{Ptac}[hGLYAT2(co_Ec)-fadD_Ec]{Placuv5}[alkLmod1], applying the protocol described in Example 12.

1-3 h after the induction, 6 g/L glycine and 6 g/L lauric acid (dissolved in lauric acid methyl ester) were added to the cultures. After a cultivation time of 48 h the entire broth of a shake flask was extracted with Acetone (ratio 1:2). Further sample treatment is described in Example 7. Samples were taken, and lauroylglycinate, lauric acid and glycine present were analysed. The results are shown in Table 14.

All strains except the none-plasmid control produced lauroylglycinate in amounts between 0.44 and 2109.8 mg/L.

TABLE 14
Quantitative determination of lauroylglycinate after a cultivation time
of 48 h in strains of E. coli W3110 ΔfadE harboring different plasmids.
Each strain was fed with 6 g/L glycine and 6 g/L lauric acid

| | | C_{lauroylglycinate} |
|---|---|---|
| strain | plasmid | [mg/L] |
| E. coli | — | 0.0 |
| W3110 | pCDF{Ptac}[hGLYAT2(co_Ec)-fadD_Ec]{Placuv5}[alkLmod1] | 2109.8 |
| ΔfadE | pCDF{Ptac}[GLYAT_Fc(co_Ec)-fadD_Ec]{Placuv5}[alkLmod1] | 1.1 |
| | pCDF{Ptac}[GLYAT_Sb(co_Ec)-fadD_Ec]{Placuv5}[alkLmod1] | 1.5 |
| | pCDF{Ptac}[GLYAT_Mm(co_Ec)-fadD_Ec]{Placuv5}[alkLmod1] | 0.5 |
| | pCDF{Ptac}[GLYAT_Nl(co_Ec)-fadD_Ec]{Placuv5}[alkLmod1] | 209.7 |
| | pCDF{Ptac}[GLYAT_Bt(co_Ec)-fadD_Ec]{Placuv5}[alkLmod1] | 0.4 |

1. A cell expressing an amino acid-N-acyl-transferase and an acyl-CoA synthetase, wherein the cell has a reduced fatty acid degradation capacity.

2. The cell according to claim 1, wherein the fatty acid degradation capacity is reduced owing to a decrease in activity, compared to a wild type cell, of at least one enzyme selected from the group consisting of acyl-CoA dehydrogenase, 2,4-dienoyl-CoA reductase, enoyl-CoA hydratase and 3-ketoacyl-CoA thiolase.

3. The cell according to claim 1, wherein the amino acid-N-acyl-transferase is a human amino acid-N-acyl-transferase.

4. The cell according to claim 1, wherein the cell is a bacterial cell.

5. The cell according to claim 1, wherein the cell is capable of making proteinogenic amino acids and/or fatty acids.

6. The cell according to claim 5, wherein the cell comprises at least one enzyme selected from the group consisting of β-ketoacyl-ACP synthase I, 3-oxoacyl-ACP-synthase I, Malonyl-CoA-ACP transacylase, enoyl ACP reductase, and β-ketoacyl-ACP synthase III capable of making proteinogenic amino acids and/or fatty acids.

7. The cell according to claim 1, wherein the cell expresses an acyl-CoA thioesterase.

8. The cell according to claim 1, wherein the acyl-CoA synthetase is SEQ ID NO: 6 or a variant thereof.

9. The cell according to claim 1, wherein the cell is genetically modified to increase the expression of at least one transporter protein compared to a wild type cell, wherein the transporter protein is selected from the group consisting of FadL and AlkL.

10. A cell expressing an amino acid-N-acyl-transferase, an acyl-CoA synthetase, and at least one enzyme selected from the group consisting of acetoacetyl-CoA synthase, ketoacyl-CoA synthase, ketoacyl-CoA thiolase, enoyl-CoA reductase, ketoacyl-CoA reductase and 3-hydroxyacyl-CoA dehydratase, wherein the cell optionally exhibits a decrease in the expression of acetoacetyl-CoA thiolase relative to a wild type cell, and wherein the cell has a reduced fatty acid degradation capacity.

11. A method for producing an acyl amino acid, comprising
contacting an amino acid and an acyl CoA in the presence of an amino acid-N-acyltransferase.

12. The method according to claim 11, further comprising
converting a fatty acid to the acyl CoA using an acyl-CoA synthetase, and/or
hydrogenating.

13. The method according to claim 12, wherein at least one enzyme selected from the group consisting of the amino acid-N-acyl-transferase and the acyl-CoA synthetase is provided in the form of a cell expressing the enzyme.

14. The method according to claim 13, wherein the cell expressing the enzyme is a cell expressing an amino acid-N-acyl-transferase and an acyl-CoA synthetase, wherein the cell has a reduced fatty acid degradation capacity.

15. A reaction mixture comprising
an amino acid-N-acyl-transferase,
an acyl-CoA synthetase,
an amino acid and
either an acyl CoA or a fatty acid and an acyl-CoA-synthetase.

16-20. (canceled)
